(19) United States
(12) Patent Application Publication
(10) Pub. No.: US 2011/0105462 A1
(43) Pub. Date: May 5, 2011

(54) MODULATORS OF DOPAMINE NEUROTRANSMISSION

(75) Inventors: Clas Sonesson, Billdal (SE); Peder Svensson, Goteborg (SE)

(73) Assignee: NSAB, FILIAL AF NEUROSEARCH SWEDEN AB, SVERIGE, Ballerup (DK)

(21) Appl. No.: 12/990,059

(22) PCT Filed: Apr. 28, 2009
(86) PCT No.: PCT/EP09/55139

§ 371 Date: Dec. 23, 2010

(30) Foreign Application Priority Data
Apr. 29, 2008 (DK) PA 2008 00599

(51) Int. Cl. A61K 31/397; C07D 319/20; C07D 405/06; C07D 413/06; A61K 31/352; A61K 31/4025; A61K 31/453; A61K 31/5377; A61P 25/16; A61P 25/14; A61P 25/00; A61P 25/28; A61P 25/22; A61P 25/24; A61P 25/30; A61P 29/00; A61P 3/04; A61P 15/00; A61P 25/18

(52) U.S. Cl. 514/210.19; 549/366; 548/526; 546/197; 548/950; 544/148; 514/452; 514/422; 514/321; 514/233.8

(57) ABSTRACT

The present invention relates to novel 1-(2,3-dihydro-1,4-benzodioxin-2-yl)-methanamine derivatives, useful as modulators of dopamine neurotransmission, and more specifically as dopaminergic stabilizers. In other aspects the invention relates to the use of these compounds in a method for therapy and to pharmaceutical compositions comprising the compounds of the invention.

### FIELD OF THE INVENTION

The present invention relates to novel 1-(2,3-dihydro-1,4-benzodioxin-2-yl)-methanamine derivatives, useful as modulators of dopamine neurotransmission, and more specifically as dopaminergic stabilizers.

In other aspects the invention relates to the use of these compounds in a method for therapy and to pharmaceutical compositions comprising the compounds of the invention.

### BACKGROUND OF THE INVENTION

Dopamine is a neurotransmitter in the brain. Since this discovery, made in the 1950's, the function of dopamine in the brain has been intensely explored. To date, it is well established that dopamine is essential in several aspects of brain function including motor, cognitive, sensory, emotional and autonomous functions (e.g. regulation of appetite, body temperature, sleep). Thus, modulation of dopaminergic function may be beneficial in the treatment of a wide range of disorders affecting brain functions. In fact, drugs that act, directly or indirectly at central dopamine receptors are commonly used in the treatment of neurological and psychiatric disorders, e.g. Parkinson's disease and schizophrenia. However, currently available dopaminergic pharmaceuticals can have severe side effects. One class of compounds acting through the dopamine systems of the brain are dopaminergic stabilizers, which have shown to be useful in the treatment of both neurologic and psychiatric disorders.

The typical pharmacological effects which are characteristic for dopaminergic stabilizers can be summarised as: 1) Increased turnover of dopamine in the terminal areas of the ascending dopaminergic projections of the mammalian brain; 2) No or only weak behavioural effects in otherwise untreated rats; and 3) Inhibition of behavioural effects induced by psychostimulants or psychotomimetic compounds in the rat. In the present invention this is referred to as a dopaminergic stabilizer profile.

### DESCRIPTION OF PRIOR ART

WO 2005/105776 discloses arylsulfonyl benzodioxanes useful as modulators of 5-HT6 and 5-HT2A receptors.

WO 2006/116158 discloses benzodioxane and benzodioxolane derivatives useful as partial agonists or agonists at 5-HT2C receptors.

Avner et al. in Journal of Medicinal Chemistry 1974 17 (2) 197-200, disclose substituted 1,4-benzodioxanes as reversible and irreversible antagonists at adrenergic receptors.

Various chlorinated 1,4-benzodioxanes have been disclosed as ligands for α1 and α2-receptors, see e.g. Timmermans et al.; Pharmacology 1983 26 (5) 258-69; Timmermans et al.; Molecular Pharmacology 1981 20 (2) 295-301; Marini-Bettolo et al.; Gazzetta Chimica Italiana 1957 87 1303-1305; Grafe et al.; Arzneimittel-Forschunq 1974 24 (2) 153-157; and Itazaki et al.; Chemical & Pharmaceutical Bulletin 1988 36 (9) 3387-403.

The compound 3-morpholin-4-ylmethyl-2,3-dihydro-benzo[1,4]dioxine-6-carbonitrile is disclosed as a synthesis intermediate by Funke et al. in Synthesis of 7-substituted-2-aminomethyl-1,4-benazodioxanes; Gazzetta Chimica Italiana 1961 91 1268-1281.

### SUMMARY OF THE INVENTION

The object of the present invention is to provide novel pharmaceutically active compounds, especially useful in treatment of disorders in the central nervous system. A further object is the provision of compounds for modulation of dopaminergic systems in the mammalian brain, including human brain. A still further object is the provision of novel compounds with a dopaminergic stabilizer profile. A further object is to provide compounds with therapeutic effects after oral administration. A still further object is the provision of compounds with more optimal pharmacodynamic properties such as e.g. kinetic behaviour, bioavailability, solubility and efficacy. A further object is to provide compounds being superior to presently known dopaminergic compounds in the treatment of several disorders related to dysfunctions of the CNS, in terms of efficacy or side effects.

The present invention concerns the unexpected discovery of the pharmacological effects of compounds of Formula 1 on the dopaminergic system in the brain. By pharmacological testing in vivo in the rat it is demonstrated that compounds of the present invention have effects on biochemical indices in the brain with the characteristic features of dopamine antagonists.

In its first aspect, the invention provides a compound of Formula 1

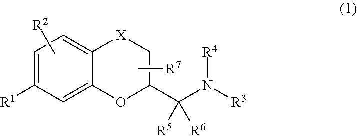

any of its stereoisomers or any mixture of its stereoisomers, or an N-oxide thereof, or a pharmaceutically acceptable salt thereof; wherein R¹, R², R³, R⁴, R⁵, R⁶, R⁷ and X are as defined below.

In its second aspect, the invention provides a pharmaceutical composition, comprising a therapeutically effective amount of a compound of the invention, any of its stereoisomers or any mixture of its stereoisomers, or an N-oxide thereof, or a pharmaceutically acceptable salt thereof, together with at least one pharmaceutically acceptable carrier, excipient or diluent.

In a further aspect, the invention provides the use of a compound of the invention, any of its stereoisomers or any mixture of its stereoisomers or an N-oxide thereof, or a pharmaceutically acceptable salt thereof, for the manufacture of a pharmaceutical composition for the treatment, prevention or alleviation of a disease or a disorder or a condition of a mammal, including a human, which disease, disorder or condition is responsive to responsive to modulation of dopaminergic function in the central nervous system.

In a still further aspect, the invention relates to a method for treatment, prevention or alleviation of a disease or a disorder or a condition of a living animal body, including a human, which disorder, disease or condition is responsive to modulation of dopaminergic function in the central nervous system, which method comprises the step of administering to such a living animal body in need thereof a therapeutically effective amount of a compound of the invention, any of its stereoisomers or any mixture of its stereoisomers, or an N-oxide thereof, or a pharmaceutically acceptable salt thereof.

Other aspects of the invention will be apparent to the person skilled in the art from the following detailed description and examples.

### DETAILED DESCRIPTION OF THE INVENTION

### 1-(2,3-Dihydro-1,4-benzodioxin-2-yl)methanamine derivatives

In its first aspect the present invention provides compounds of Formula 1:

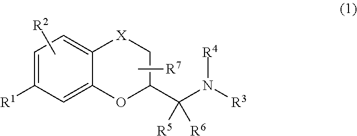

any of its stereoisomers or any mixture of its stereoisomers, or an N-oxide thereof, or a pharmaceutically acceptable salt thereof, wherein

X is O, S, NH or CH₂;

R¹ is selected from the group consisting of OSO₂CF₃, OSO₂CH₃, COR⁸, CN, OCF₃, SCF₃, OCHF₂, SCHF₂, CF₃, F, Cl, Br, I, SF₅, SCN, OCN, OCOCF₃, SCOCF₃, OCOCH₃, SCOCH₃ and CH(OH)CF₃;

R₂ is selected from the group consisting of H, CN, F, Cl, Br, I and CH₃;

R₃ is selected from the group consisting of C₁-C₅ alkyl, allyl, CH₂CH₂OCH₃, CH₂CH₂CH₂F, CH₂CH₂CHF₂, CH₂CH₂F, 3,3,3-trifluoropropyl, 4,4,4-trifluorobutyl, CH₂CH₂OH, CH₂CH₂CH₂OH, CH₂CH(OH)CH₃, CH₂CH₂COCH₃, C₃-C₆ cycloalkyl,

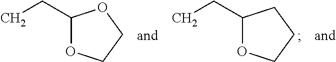

R⁴ is selected from the group consisting of H and C₁-C₅ alkyl; or

R³ and R⁴ together with the nitrogen atom to which they are attached form a four- to six-membered heterocyclic ring, which heterocyclic ring may optionally comprise as a ring member, one oxygen atom, and/or one additional nitrogen atom; and which heterocyclic ring may optionally be substituted with C₁-C₅ alkyl; and

R⁵, R⁶ and R⁷ are selected from the group consisting of H and CH₃;

R⁸ is selected from the group consisting of C₁-C₃ alkyl, CF₃, CHF₂, CH₂F and CN.

In a more preferred embodiment the compound of the invention is a compound of Formula 1A:

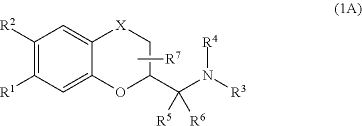

any of its stereoisomers or any mixture of its stereoisomers, or an N-oxide thereof, or a pharmaceutically acceptable salt thereof, wherein X, R¹, R², R³, R⁴, R⁵, R⁶ and R⁷ are as above.

In another more preferred embodiment the compound of the invention is a compound of Formula 1B:

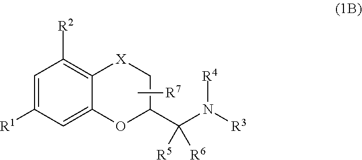

any of its stereoisomers or any mixture of its stereoisomers, or an N-oxide thereof, or a pharmaceutically acceptable salt thereof, wherein X, R¹, R², R³, R⁴, R⁵, R⁶ and R⁷ are as defined above.

In a third more preferred embodiment the compound of the invention is a compound of Formula 1C:

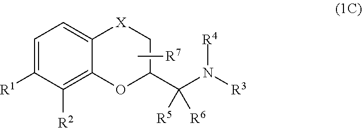

any of its stereoisomers or any mixture of its stereoisomers, or an N-oxide thereof, or a pharmaceutically acceptable salt thereof, wherein X, R¹, R², R³, R⁴, R⁵, R⁶ and R⁷ are as defined above.

In a preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein X is O, S, NH or CH₂.

In a more preferred embodiment X is O.

In another more preferred embodiment X is S.

In a third more preferred embodiment X is NH.

In a fourth more preferred embodiment X is CH₂.

In another preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R¹ is selected from the group consisting of OSO₂CF₃, OSO₂CH₃, COR⁸, CN, OCF₃, SCF₃, OCHF₂, SCHF₂, CF₃, F, Cl, Br, I, SF₅, SCN, OCN, OCOCF₃, SCOCF₃, OCOCH₃, SCOCH₃ and CH(OH)CF₃; and R⁸ is selected from the group consisting of C₁-C₃ alkyl, CF₃, CHF₂, CH₂F and CN.

In a more preferred embodiment R¹ is OSO₂CF₃.

In another more preferred embodiment R¹ is COR⁸; and R⁸ is selected from the group consisting of C₁-C₃ alkyl, CF₃, CHF₂, CH₂F and CN.

In a third more preferred embodiment R¹ is CN

In a fourth more preferred embodiment R¹ is OCF₃.

In a fifth more preferred embodiment R¹ is SCF₃.

In a sixth more preferred embodiment R¹ is OCHF₂.

In a seventh more preferred embodiment R¹ is SCHF₂.

In an eight more preferred embodiment R¹ is CF₃.

In a ninth more preferred embodiment R¹ is F.

In a tenth more preferred embodiment R¹ is Cl.

In an eleventh more preferred embodiment R¹ is Cl; and with the proviso that R⁴ is H.

In an twelfth more preferred embodiment R¹ is Br.

In a thirteenth more preferred embodiment R¹ is I.

In a fourteenth more preferred embodiment R¹ is SF₅.

In a fifteenth more preferred embodiment R¹ is SCN.

In a sixteenth more preferred embodiment R¹ is OCN.

In a seventeenth more preferred embodiment R¹ is OCN, OCOCF₃.

In a eighteenth more preferred embodiment R¹ is OCOCF₃.

In an nineteenth more preferred embodiment R¹ is SCOCF₃.

In a twentieth more preferred embodiment R¹ is OCOCH₃.

In a twentyfirst more preferred embodiment R¹ is SCOCH₃.

In a twentysecond more preferred embodiment R¹ is CH(OH)CF₃.

In a twentythird more preferred embodiment R¹ is selected from the group consisting of CF₃, OSO₂CH₃ and OSO₂CF₃.

In a twentyfourth more preferred embodiment R¹ is selected from the group consisting F and Br.

In a third preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R² is selected from the group consisting of H, CN, F, Cl, Br, I and CH₃.

In a more preferred embodiment R² is H.

In another more preferred embodiment R² is CN.

In a third more preferred embodiment R² is F.

In a fourth more preferred embodiment R² is Cl

In a fifth more preferred embodiment R² is Br.

In a sixth more preferred embodiment R² is I.

In a seventh more preferred embodiment R² is CH₃.

In an eight more preferred embodiment R² is selected from the group consisting of H, F and Cl.

In a ninth more preferred embodiment R² is H or F.

In a fourth preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R³ is selected from the group consisting of C₁-C₅ alkyl, allyl, CH₂CH₂OCH₃, CH₂CH₂CH₂F, CH₂CH₂CHF₂, CH₂CH₂F, 3,3,3-trifluoropropyl, 4,4,4-trifluorobutyl, CH₂CH₂OH, CH₂CH₂CH₂OH, CH₂CH(OH)CH₃, CH₂CH₂COCH₃, C₃-C₆ cycloalkyl,

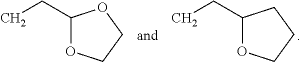

In a more preferred embodiment R³ is C₁-C₅ alkyl.

In another more preferred embodiment R³ is allyl.

In a third more preferred embodiment R³ is CH₂CH₂OCH₃.

In a fourth more preferred embodiment R³ is CH₂CH₂CH₂F.

In a fifth more preferred embodiment R³ is CH₂CH₂CHF₂.

In a sixth more preferred embodiment R³ is CH₂CH₂F.

In a seventh more preferred embodiment R³ is 3,3,3-trifluoropropyl.

In an eight more preferred embodiment R³ is 4,4,4-trifluorobutyl.

In a ninth more preferred embodiment R³ is CH₂CH₂OH.

In a tenth more preferred embodiment R³ is CH₂CH₂CH₂OH.

In an eleventh more preferred embodiment R³ is CH₂CH(OH)CH₃.

In a twelfth more preferred embodiment R³ is CH₂CH₂COCH₃.

In a thirteenth more preferred embodiment R³ is C₃-C₆ cycloalkyl.

In a fourteenth more preferred embodiment R³ is

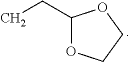

In a fifteenth more preferred embodiment R³ is

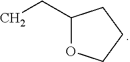

In a sixteenth more preferred embodiment R³ is selected from the group consisting of C₁-C₅ alkyl, allyl, CH₂CH₂OCH₃ and CH₂CH₂OH.

In a seventeenth more preferred embodiment R³ is selected from the group consisting of C₁-C₅ alkyl, allyl and CH₂CH₂OH.

In a fifth preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R⁴ is selected from the group consisting of H and C₁-C₅ alkyl.

In a more preferred embodiment R⁴ is H.

In a another more preferred embodiment R⁴ is H; and with the proviso that R¹ is Cl.

In a third more preferred embodiment R⁴ is C₁-C₅ alkyl.

In a fourth more preferred embodiment R⁴ is selected from the group consisting of H and C₁-C₅ alkyl.

In a sixth preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R³ and R⁴ together with the nitrogen atom to which they are attached form a four- to six-membered heterocyclic ring, which heterocyclic ring may optionally comprise as a ring member, one oxygen atom, and/or one additional nitrogen atom; and which heterocyclic ring may optionally be substituted with C₁-C₅ alkyl.

In a more preferred embodiment R³ and R⁴ together with the nitrogen atom to which they are attached form a four- to six-membered heterocyclic ring.

In another more preferred embodiment R³ and R⁴ together the nitrogen atom to which they are attached form acetidine, pyrrolidine, piperidine, C₁-C₅ alkyl-piperidine or morpholine.

In a third more preferred embodiment R³ and R⁴ together the nitrogen atom to which they are attached form an acetidine, a pyrrolidine, a piperidine or a morpholine group.

In a fourth more preferred embodiment R³ and R⁴ together the nitrogen atom to which they are attached form an acetidine group.

In a fifth more preferred embodiment R³ and R⁴ together the nitrogen atom to which they are attached form a pyrrolidine group.

In a sixth more preferred embodiment R³ and R⁴ together the nitrogen atom to which they are attached form a piperidine group.

In a seventh more preferred embodiment R³ and R⁴ together the nitrogen atom to which they are attached form a C₁-C₅ alkyl-piperidine group.

In an eight more preferred embodiment R³ and R⁴ together the nitrogen atom to which they are attached form a morpholine group.

In a seventh preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein R⁵, R⁶ and R⁷ are selected from the group consisting of H and CH₃.

In a more preferred embodiment each of R⁵, R⁶ and R⁷ is H.

In an eight preferred embodiment the compound of the invention is a compound of Formula 1, 1A, 1B or 1C, any of its stereoisomers or any mixture of its stereoisomers, or an N-oxide thereof, or a pharmaceutically acceptable salt thereof, wherein

X is O;

R¹ is OSO₂CF₃, OSO₂CH₃, CF₃, F, Cl, Br; and with the proviso that R⁴ is H if R¹ is Cl;

R² is H, F;

R³ is C₁-C₅ alkyl, allyl or CH₂CH₂OH; and

R⁴ is H and C₁-C₅ alkyl; and with the proviso that R¹ is Cl if R⁴ is H; or

R³ and R⁴ together the nitrogen atom to which they are attached form an acetidine, a pyrrolidine, a piperidine or a morpholine group; and

R⁵, R⁶ and R⁷ are selected from the group consisting of H and CH₃.

In a yet more preferred embodiment the compound of the invention is
N-{[(2S)-7-BROMO-2,3-DIHYDRO-1,4-BENZODIOXIN-2-YL]METHYL}PROPAN-1-AMINE;
N-[(6,7-DIFLUORO-2,3-DIHYDRO-1,4-BENZODIOXIN-2-YL)METHYL]PROPAN-1-AMINE;
N-[(7-FLUORO-2,3-DIHYDRO-1,4-BENZODIOXIN-2-YL)METHYL]PROPAN-1-AMINE;
N-[(7-CHLORO-2,3-DIHYDRO-1,4-BENZODIOXIN-2-YL)METHYL]PROPAN-1-AMINE;
3-[(PROPYLAMINO)METHYL]-2,3-DIHYDRO-1,4-BENZODIOXIN-6-YL METHANESULFONATE;
3-[(PROPYLAMINO)METHYL]-2,3-DIHYDRO-1,4-BENZODIOXIN-6-YL TRIFLUOROMETHANESULFONATE;
N-[(7,8-DIFLUORO-2,3-DIHYDRO-1,4-BENZODIOXIN-2-YL)METHYL]PROPAN-1-AMINE;
N-[(5,7-DIFLUORO-2,3-DIHYDRO-1,4-BENZODIOXIN-2-YL)METHYL]PROPAN-1-AMINE;
1-{[(2S)-7-FLUORO-2,3-DIHYDRO-1,4-BENZODIOXIN-2-YL]METHYL}PYRROLIDINE;
1-[(7-FLUORO-2,3-DIHYDRO-1,4-BENZODIOXIN-2-YL)-N-METHYLMETHANAMINE;
N-[(7-FLUORO-2,3-DIHYDRO-1,4-BENZODIOXIN-2-YL)METHYL]BUTAN-1-AMINE;
2-{[(7-FLUORO-2,3-DIHYDRO-1,4-BENZODIOXIN-2-YL)METHYL]AMINO}ETHANOL;
N-[(7-FLUORO-2,3-DIHYDRO-1,4-BENZODIOXIN-2-YL)METHYL]-N-PROPYLPROPAN-1-AMINE;
N-ETHYL-N-[(7-FLUORO-2,3-DIHYDRO-1,4-BENZODIOXIN-2-YL)METHYL]PROPAN-1-AMINE;
1-[(7-FLUORO-2,3-DIHYDRO-1,4-BENZODIOXIN-2-YL)METHYL]PIPERIDINE;
N-[(7-FLUORO-2,3-DIHYDRO-1,4-BENZODIOXIN-2-YL)METHYL]ETHANAMINE;
N-[(7-FLUORO-2,3-DIHYDRO-1,4-BENZODIOXIN-2-YL)METHYL]PROP-2-EN-1-AMINE;
1-[(7-FLUORO-2,3-DIHYDRO-1,4-BENZODIOXIN-2-YL)-N,N-DIMETHYLMETHANAMINE;
N-[(7-FLUORO-2,3-DIHYDRO-1,4-BENZODIOXIN-2-YL)METHYL]-N-METHYLPROPAN-1-AMINE;
1-[(7-FLUORO-2,3-DIHYDRO-1,4-BENZODIOXIN-2-YL)METHYL]AZETIDINE;
4-[(7-FLUORO-2,3-DIHYDRO-1,4-BENZODIOXIN-2-YL)METHYL]MORPHOLINE;
N-[(7-FLUORO-2,3-DIHYDRO-1,4-BENZODIOXIN-2-YL)METHYL]-2-METHOXYETHANAMINE;
N-ETHYL-N-[(7-FLUORO-2,3-DIHYDRO-1,4-BENZODIOXIN-2-YL)METHYL]ETHANAMINE;
N-[(7-FLUORO-2,3-DIHYDRO-1,4-BENZODIOXIN-2-YL)METHYL]-N-METHYLETHANAMINE;
3-[(METHYLAMINO)METHYL]-2,3-DIHYDRO-1,4-BENZODIOXIN-6-YL METHANESULFONATE;
3-[(ETHYLAMINO)METHYL]-2,3-DIHYDRO-1,4-BENZODIOXIN-6-YL METHANESULFONATE;
3-[(BUTYLAMINO)METHYL]-2,3-DIHYDRO-1,4-BENZODIOXIN-6-YL METHANESULFONATE;
3-{[(2-HYDROXYETHYL)AMINO]METHYL}-2,3-DIHYDRO-1,4-BENZODIOXIN-6-YL METHANESULFONATE;
3-[(DIPROPYLAMINO)METHYL]-2,3-DIHYDRO-1,4-BENZODIOXIN-6-YL METHANESULFONATE;
3-{[ETHYL(PROPYL)AMINO]METHYL}-2,3-DIHYDRO-1,4-BENZODIOXIN-6-YL METHANESULFONATE;
3-(PIPERIDIN-1-YLMETHYL)-2,3-DIHYDRO-1,4-BENZODIOXIN-6-YL METHANESULFONATE;
3-[(DIMETHYLAMINO)METHYL]-2,3-DIHYDRO-1,4-BENZODIOXIN-6-YL METHANESULFONATE;
3-{[METHYL(PROPYL)AMINO]METHYL}-2,3-DIHYDRO-1,4-BENZODIOXIN-6-YL METHANESULFONATE;
3-(MORPHOLIN-4-YLMETHYL)-2,3-DIHYDRO-1,4-BENZODIOXIN-6-YL METHANESULFONATE;
3-[(DIETHYLAMINO)METHYL]-2,3-DIHYDRO-1,4-BENZODIOXIN-6-YL METHANESULFONATE;
3-(PYRROLIDIN-1-YLMETHYL)-2,3-DIHYDRO-1,4-BENZODIOXIN-6-YL METHANESULFONATE;
3-[(ALLYLAMINO)METHYL]-2,3-DIHYDRO-1,4-BENZODIOXIN-6-YL METHANESULFONATE;
3-{[ETHYL(METHYL)AMINO]METHYL}-2,3-DIHYDRO-1,4-BENZODIOXIN-6-YL METHANESULFONATE;
3-{[(2-METHOXYETHYL)AMINO]METHYL}-2,3-DIHYDRO-1,4-BENZODIOXIN-6-YL METHANESULFONATE;
3-(AZETIDIN-1-YLMETHYL)-2,3-DIHYDRO-1,4-BENZODIOXIN-6-YL METHANESULFONATE;
3-[(METHYLAMINO)METHYL]-2,3-DIHYDRO-1,4-BENZODIOXIN-6-YL TRIFLUOROMETHANESULFONATE;
3-[(ETHYLAMINO)METHYL]-2,3-DIHYDRO-1,4-BENZODIOXIN-6-YL TRIFLUOROMETHANESULFONATE;
3-[(BUTYLAMINO)METHYL]-2,3-DIHYDRO-1,4-BENZODIOXIN-6-YL TRIFLUOROMETHANESULFONATE;
3-{[(2-HYDROXYETHYL)AMINO]METHYL}-2,3-DIHYDRO-1,4-BENZODIOXIN-6-YL TRIFLUOROMETHANESULFONATE;
3-[(DIPROPYLAMINO)METHYL]-2,3-DIHYDRO-1,4-BENZODIOXIN-6-YL TRIFLUOROMETHANESULFONATE;
3-{[ETHYL(PROPYL)AMINO]METHYL}-2,3-DIHYDRO-1,4-BENZODIOXIN-6-YL TRIFLUOROMETHANESULFONATE;
3-(PIPERIDIN-1-YLMETHYL)-2,3-DIHYDRO-1,4-BENZODIOXIN-6-YL TRIFLUOROMETHANESULFONATE;
3-[(DIMETHYLAMINO)METHYL]-2,3-DIHYDRO-1,4-BENZODIOXIN-6-YL TRIFLUOROMETHANESULFONATE;
3-(MORPHOLIN-4-YLMETHYL)-2,3-DIHYDRO-1,4-BENZODIOXIN-6-YL TRIFLUOROMETHANESULFONATE;
3-[(DIETHYLAMINO)METHYL]-2,3-DIHYDRO-1,4-BENZODIOXIN-6-YL TRIFLUOROMETHANESULFONATE;
3-(PYRROLIDIN-1-YLMETHYL)-2,3-DIHYDRO-1,4-BENZODIOXIN-6-YL TRIFLUOROMETHANESULFONATE;
3-[(ALLYLAMINO)METHYL]-2,3-DIHYDRO-1,4-BENZODIOXIN-6-YL TRIFLUOROMETHANESULFONATE;
3-{[ETHYL(METHYL)AMINO]METHYL}-2,3-DIHYDRO-1,4-BENZODIOXIN-6-YL TRIFLUOROMETHANESULFONATE;
3-{[(2-METHOXYETHYL)AMINO]METHYL}-2,3-DIHYDRO-1,4-BENZODIOXIN-6-YL TRIFLUOROMETHANESULFONATE;
3-(AZETIDIN-1-YLMETHYL)-2,3-DIHYDRO-1,4-BENZODIOXIN-6-YL TRIFLUOROMETHANESULFONATE;
3-{[METHYL(PROPYL)AMINO]METHYL}-2,3-DIHYDRO-1,4-BENZODIOXIN-6-YL TRIFLUOROMETHANESULFONATE;
1-[(6,7-DIFLUORO-2,3-DIHYDRO-1,4-BENZODIOXIN-2-YL)METHYL]PYRROLIDINE;
N-{[(2S)-7-(TRIFLUOROMETHYL)-2,3-DIHYDRO-1,4-BENZODIOXIN-2-YL]METHYL}PROPAN-1-AMINE;
1-(5,7-DIFLUORO-2,3-DIHYDRO-1,4-BENZODIOXIN-2-YL)-N-METHYLMETHANAMINE;
N-[(5,7-DIFLUORO-2,3-DIHYDRO-1,4-BENZODIOXIN-2-YL)METHYL]ETHANAMINE;
N-[(5,7-DIFLUORO-2,3-DIHYDRO-1,4-BENZODIOXIN-2-YL)METHYL]PROP-2-EN-1-AMINE;
4-[(5,7-DIFLUORO-2,3-DIHYDRO-1,4-BENZODIOXIN-2-YL)METHYL]MORPHOLINE;
N-[(5,7-DIFLUORO-2,3-DIHYDRO-1,4-BENZODIOXIN-2-YL)METHYL]BUTAN-1-AMINE;
N-[(5,7-DIFLUORO-2,3-DIHYDRO-1,4-BENZODIOXIN-2-YL)METHYL]-N-PROPYLPROPAN-1-AMINE;
1-(5,7-DIFLUORO-2,3-DIHYDRO-1,4-BENZODIOXIN-2-YL)-N,N-DIMETHYLMETHANAMINE;
N-[(5,7-DIFLUORO-2,3-DIHYDRO-1,4-BENZODIOXIN-2-YL)METHYL]-N-ETHYLETHANAMINE;
N-[(5,7-DIFLUORO-2,3-DIHYDRO-1,4-BENZODIOXIN-2-YL)METHYL]PROPAN-2-AMINE;
N-[(5,7-DIFLUORO-2,3-DIHYDRO-1,4-BENZODIOXIN-2-YL)METHYL]-N-METHYLPROPAN-1-AMINE;
N-[(5,7-DIFLUORO-2,3-DIHYDRO-1,4-BENZODIOXIN-2-YL)METHYL]-N-ETHYLPROPAN-1-AMINE;
2-{[(5,7-DIFLUORO-2,3-DIHYDRO-1,4-BENZODIOXIN-2-YL)METHYL]AMINO}ETHANOL;
N-[(5,7-DIFLUORO-2,3-DIHYDRO-1,4-BENZODIOXIN-2-YL)METHYL]-N-METHYLETHANAMINE;
N-[(5,7-DIFLUORO-2,3-DIHYDRO-1,4-BENZODIOXIN-2-YL)METHYL]-2-METHOXYETHANAMINE;
1-[(5,7-DIFLUORO-2,3-DIHYDRO-1,4-BENZODIOXIN-2-YL)METHYL]AZETIDINE;
N-[(5,7-DIFLUORO-2,3-DIHYDRO-1,4-BENZODIOXIN-2-YL)METHYL]-2-METHYLPROPAN-1-AMINE;
1-[(5,7-DIFLUORO-2,3-DIHYDRO-1,4-BENZODIOXIN-2-YL)METHYL]PYRROLIDINE;
1-[(5,7-DIFLUORO-2,3-DIHYDRO-1,4-BENZODIOXIN-2-YL)METHYL]PIPERIDINE;
N-[(5,7-DIFLUORO-2,3-DIHYDRO-1,4-BENZODIOXIN-2-YL)METHYL]-3-FLUOROPROPAN-1-AMINE;
2-({[(2S)-7-(TRIFLUOROMETHYL)-2,3-DIHYDRO-1,4-BENZODIOXIN-2-YL]METHYL}AMINO)ETHANOL; or
N-{[7-(FLUOROMETHYLSULFONYL)-3,4-DIHYDRO-2H-CHROMEN-2-YL]METHYL}-N-PROPAN-1-AMINE;

any of its stereoisomers or any mixture of its stereoisomers, or an N-oxide thereof, or a pharmaceutically acceptable salt thereof.

Any combination of two or more of the embodiments as described above is considered within the scope of the present invention.

### DEFINITION OF SUBSTITUENTS

In the context of this invention C₁-C₅ alkyl means a straight chain or branched chain of one to five carbon atoms, including but not limited to, methyl, ethyl, n-propyl, i-propyl, n-butyl, i-butyl, s-butyl, t-butyl, n-pentyl, i-pentyl, neo-pentyl.

C₃-C₆ cycloalkyl designates a cyclic alkyl group containing of from three to six carbon atoms, including cyclopropyl, cyclobutyl and cyclopentyl.

The term “allyl” refers to the group —CH₂—CH═CH₂.

Four- to six-membered heterocyclic rings comprising at least one nitrogen atom include for example, but not limited to, acetidine, pyrrolidine, piperidine and morpholine.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may exist in different stereoisomeric forms—including enantiomers, diastereomers or cis-trans-isomers.

The invention includes all such isomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphor-sulphonate) salts for example.

The chemical compounds of the present invention may also be resolved by the formation of diastereomeric amides by reaction of the chemical compounds of the present invention with an optically active carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### N-Oxides

In the context of this invention an N-oxide designates an oxide derivative of a tertiary amine, including a nitrogen atom of an aromatic N-heterocyclic compound, a non-aromatic N-heterocyclic compounds, a trialkylamine and a trialkenylamine. For example, the N-oxide of a compound containing a pyridyl may be the 1-oxy-pyridin-2, -3 or -4-yl derivative.

N-oxides of the compounds of the invention may be prepared by oxidation of the corresponding nitrogen base using a conventional oxidizing agent such as hydrogen peroxide in the presence of an acid such as acetic acid at an elevated temperature, or by reaction with a peracid such as peracetic acid in a suitable solvent, e.g. dichloromethane, ethyl acetate or methyl acetate, or in chloroform or dichloromethane with 3-chloroperoxybenzoic acid.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radio-nuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

Persons skilled in the art will appreciate that, in order to obtain compounds of the invention in an alternative—and in some occasions, more convenient manner—the individual process steps mentioned hereinbefore may be performed in a different order, and/or the individual reactions may be performed at different stage in the overall route (i.e. chemical transformations may be performed upon different intermediates to those associated hereinbefore with a particular reaction).

### Biological Activity

The typical pharmacological effects which are characteristic for dopaminergic stabilizers are an increased turnover of dopamine in the terminal areas of the ascending dopaminergic projections of the mammalian brain. This can be illustrated by measuring of changes in biochemical indices in the brain with the characteristic features of dopamine antagonists, e.g. producing increases in concentrations of dopamine metabolites such as 3,4-dihydroxyphenyl-acetic acid (DOPAC) in the striatum. The typical increase in DOPAC levels (striatum) possible to achieve is in the range of 350-400% of control.

Representative compounds of the invention are shown in Table 1.

TABLE 1
Estimated ED₅₀ values on increase of DOPAC (3,4-
dihydroxyphenylacetic acid) in the rat striatum after systemic
adminstration of test compound. For methods and statistical
calculations see the enclosed tests.

| | ED₅₀ DOPAC* |
|---|---|
| Examples | μmol/kg |
| Example 1 | 3.4 |
| | (2.6-4.1) |
| Example 6 | 12 |
| | (10-16) |
| Example 7 | 3.1 |
| | (2.1-4.0) |
| Example 8 | 14 |
| | (9.2-21) |
| Example 58 | <3.7 |

The compounds according to the present invention possess dopamine-modulating properties and both they and their pharmaceutical compositions are useful in treating numerous central nervous system disorders, including both psychiatric and neurological disorders. Particularly, the compounds and their pharmaceutical compositions may be used in the treatment of CNS disorders where the dopaminergic system is dysfunctional due to direct or indirect causes.

The compounds and compositions according to the invention can be used to improve all forms of psychosis, including schizophrenia and schizophreniform and bipolar disorders as well as drug induced psychotic disorders. Iatrogenic psychoses and hallucinoses and non-iatrogenic psychoses and hallucinoses may also be treated.

In a preferred embodiment the disease, disorder or condition contemplated according to the invention is a form of psychosis, in particular schizophrenia, a schizophreniform disorder, a bipolar disorder, or a drug induced psychotic disorder.

Mood and anxiety disorders, depression and obsessive-compulsive disease may also be treated with the compounds and compositions according to the invention.

Compounds with modulating effects on dopaminergic systems may also be used to improve motor and cognitive functions and in the treatment of emotional disturbances related to ageing, neurodegenerative (e.g. dementia and age-related cognitive impairment) and developmental disorders (such as Autism spectrum disorders, ADHD, Cerebral Palsy, Gilles de la Tourette's syndrome) as well as after brain injury. Such brain injury may be induced by traumatic, inflammatory, infectious, neoplastic, vascular, hypoxic or metabolic causes or by toxic reactions to exogenous chemicals, wherein the exogenous chemicals are selected from the group consisting of substances of abuse, pharmaceutical compounds and environmental toxins

The compounds and pharmaceutical compositions according to the invention may also be used in behavioural disorders usually first diagnosed in infancy, childhood, or adolescence as well as in impulse control disorders.

They can also be used for treating substance abuse disorders as well as disorders characterized by misuse of food. They are further useful for treatment of a condition selected from the group consisting of sleep disorders, sexual disorders, eating disorders, obesitas, and headaches and other pains in conditions characterized by increased muscular tone.

Neurological indications include the use of the compounds and their pharmaceutical compositions to improve mental and motor function in Parkinson's disease, and in related parkinsonian syndromes, dyskinesias (including L-DOPA induced dyskinesias) and dystonias. They may also be used to ameliorate tics and tremor of different origins. Moreover, they may be used to relieve pain in conditions characterized by increased muscle tone.

They can also be used in the treatment of Huntington's disease and other movement disorders as well as movement disorders induced by drugs. Restless legs and related disorders as well as narcolepsy may also be treated with compounds included according to the invention.

The compounds and their pharmaceutical compositions according to the present invention can be used for the treatment of Alzheimer's disease or related dementia disorders.

The effects of compounds of the invention on spontaneous locomotion is shown in Table 2.

TABLE 2
Effects of compounds from the present invention on Locomotor activity
in drug-naive rats. The animals were placed in the motility meters
immediately after drug administration and locomotor activity was
recorded for 60 minutes (counts/60 min ± SEM).

| Example | Control group | 3.7 μmol/kg | 11 μmol/kg | 33 μmol/kg |
|---|---|---|---|---|
| Example 1 | 8702 ± 1053 | 6714 ± 601 | 5111 ± 620 | 1326 ± 131 |
| Example 6 | 10941 ± 2360 | 9495 ± 2036 | 10705 ± 1903 | 8064 ± 707 |
| Example 7 | 6996 ± 1381 | 7932 ± 2067 | 6884 ± 400 | 4408 ± 212 |
| Example 8 | 8318 ± 2419 | 9266 ± 2706 | 8320 ± 2405 | 5457 ± 536 |
| Example 9 | 11544 ± 3670 | 5419 ± 1414 | 2769 ± 647 | 1944 ± 482 |

The effects of compounds of the invention on the increase in activity induced by direct or indirect dopaminergic agonists, i.e. d-amphetamine and congeners are shown in Table 3.

TABLE 3
Effect of compound of the present invention on reduction of
amphetamine-induced hyper-locomotion. For methods and
statistical calculations see the enclosed tests.

| | ED₅₀ |
|---|---|
| Example | μmol/kg |
| Example 7 | 13 |
| | (10-15) |

### Pharmaceutical Compositions

In another aspect the invention provides novel pharmaceutical compositions comprising a therapeutically effective amount of the chemical compound of the invention.

The present invention relates to pharmaceutical compositions comprising the compounds of the present invention, and their use in treating CNS disorders. Both organic and inorganic acids can be employed to form non-toxic pharmaceutically acceptable acid addition salts of the compounds according to the invention. Suitable acid addition salts of the compounds of the present invention include those formed with pharmaceutically acceptable salts such as those mentioned above. The pharmaceutical composition comprising a compound according to the invention may also comprise substances used to facilitate the production of the pharmaceutical preparation or the administration of the preparations. Such substances are well known to people skilled in the art and may for instance be pharmaceutically acceptable adjuvants, carriers and preservatives.

In clinical practice, the compounds according to the present invention will normally be administered orally, rectally, nasally or by injection, in the form of pharmaceutical preparations comprising the active ingredient either as a free base or as a pharmaceutically acceptable non-toxic, acid addition salt, such as the hydrochloride, lactate, acetate or sulfamate salt, in association with a pharmaceutically acceptable carrier. The carrier may be a solid, semisolid or liquid preparation. Usually the active substance will constitute between 0.1 and 99% by weight of the preparation, more specifically between 0.5 and 20% by a weight for preparations intended for injection and between 0.2 and 50% by weight for preparations suitable for oral administration.

To produce pharmaceutical preparations containing the compound according to the invention in the form of dosage units for oral application, the selected compound may be mixed with a solid excipient, e.g. lactose, saccharose, sorbitol, mannitol, starches such as potato starch, corn starch or amylopectin, cellulose derivatives, a binder such as gelatine or polyvinyl-pyrrolidine, and a lubricant such as magnesium stearate, calcium stearate, polyethylene glycol, waxes, paraffin, and the like, and then compressed into tablets. If coated tablets are required, the cores (prepared as described above) may be coated with a concentrated sugar solution which may contain e.g. gum arabic, gelatine, talcum, titanium dioxide, and the like. Alternatively, the tablet can be coated with a polymer known to the man skilled in the art, dissolved in a readily volatile organic solvent or mixture of organic solvents. Dyestuffs may be added to these coatings in order to readily distinguish between tablets containing different active substances or different amounts of the active compound.

For the preparation of soft gelatine capsules, the active substance may be admixed with e.g. a vegetable oil or polyethylene glycol. Hard gelatine capsules may contain granules of the active substance using either the mentioned excipients for tablets e.g. lactose, saccharose, sorbitol, mannitol, starches (e.g. potato starch, corn starch or amylopectin), cellulose derivatives or gelatine. Also liquids or semisolids of the drug can be filled into hard gelatine capsules.

Examples of tablet and capsule formulations suitable for oral administration are given below:

| Tablet I | mg/tablet |
|---|---|
| Compound | 100 |
| Lactose Ph. Eur | 182.75 |
| Croscarmellose sodium | 12.0 |
| Maize starch paste (5% w/v paste) | 2.25 |
| Magnesium stearate | 3.0 |

| Tablet II | mg/tablet |
|---|---|
| Compound | 50 |
| Lactose Ph. Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

| Tablet III | mg/tablet |
|---|---|
| Compound | 1.0 |
| Lactose Ph. Eur | 93.25 |
| Croscarmellose sodium | 4.0 |
| Maize starch paste (5% w/v paste) | 0.75 |
| Magnesium stearate | 1.0 |

| Capsule | mg/capsule |
|---|---|
| Compound | 10 |
| Lactose Ph. Eur | 488.5 |
| Magnesium | 1.5 |

Dosage units for rectal application can be solutions or suspensions or can be prepared in the form of suppositories comprising the active substance in a mixture with a neutral fatty base, or gelatine rectal capsules comprising the active substance in admixture with vegetable oil or paraffin oil. Liquid preparations for oral application may be in the form of syrups or suspensions, for example solutions containing from about 0.2% to about 20% by weight of the active substance herein described, the balance being sugar and mixture of ethanol, water, glycerol and propylene glycol. Optionally such liquid preparations may contain coloring agents, flavoring agents, saccharine and carboxymethylcellulose as a thickening agent or other excipients known to the man in the art.

Solutions for parenteral applications by injection can be prepared in an aqueous solution of a water-soluble pharmaceutically acceptable salt of the active substance, preferably in a concentration of from 0.5% to about 10% by weight. These solutions may also containing stabilizing agents and/or buffering agents and may conveniently be provided in various dosage unit ampoules. The use and administration to a patient to be treated would be readily apparent to an ordinary skill in the art.

For intranasal administration or administration by inhalation, the compounds of the present invention may be delivered in the form of a solution, dry powder or suspension. Administration may take place via a pump spray container that is squeezed or pumped by the patient or through an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. The compounds of the invention may also be administered via a dry powder inhaler, either as a finely divided powder in combination with a carrier substance (e.g. a saccharide) or as microspheres. The inhaler, pump spray or aerosol spray may be single or multi dose. The dosage may be controlled through a valve that delivers a measured amount of active compound.

The compounds of the invention may also be administered in a controlled release formulation. The compounds are released at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. The compounds may also be formulated in controlled release formulations in which release of the active compound is targeted. For example, release of the compound may be limited to a specific region of the digestive system through the pH sensitivity of the formulation. Such formulations are well known to persons skilled in the art.

Further details on techniques for formulation and administration may be found in the latest edition of Remington's Pharmaceutical Sciences (Maack Publishing Co., Easton, Pa.).

Depending upon the disorder and patient to be treated and the route of administration, the compositions may be administered at varying doses. The dosing will also depend upon the relation of potency to absorbability and the frequency and route of administration. Such doses may be administered once, twice or three or more times daily. The compounds of this invention can be administered to subjects in doses ranging from 0.01 mg to 500 mg per kg of body weight per day, although variations will necessarily occur depending upon the weight, sex and condition of the subject being treated, the disease state being treated and the particular route of administration chosen. However, a dosage level that is in the range of from 0.1 mg to 10 mg per kg of body weight per day, single or divided dosage is most desirably employed in humans for the treatment of diseases. Alternatively, the dosage level is such that a serum concentration of between 0.1 nM to 10 μM of the compound is obtained.

### EXAMPLES

The invention is further illustrated in the examples below and as outlined below in Schemes 1-3, which in no way are intended to limit the scope of the invention.

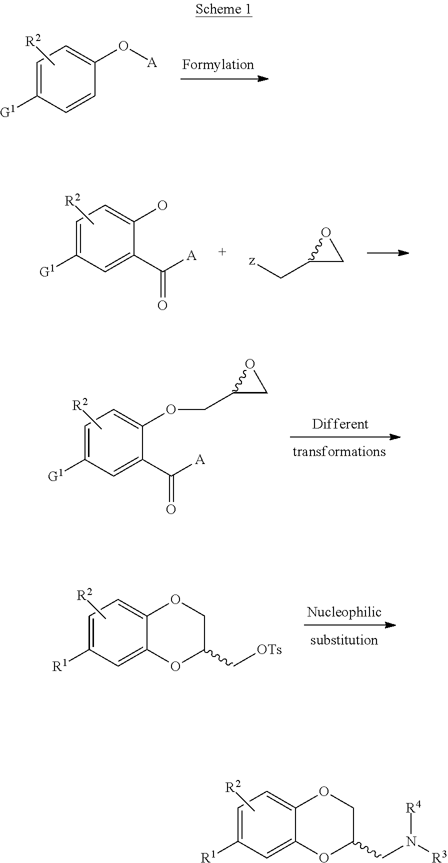

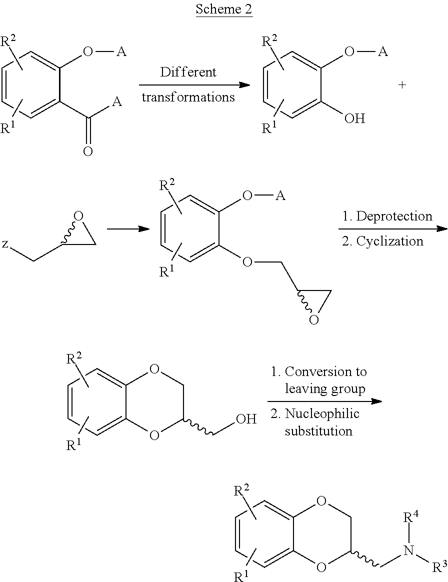

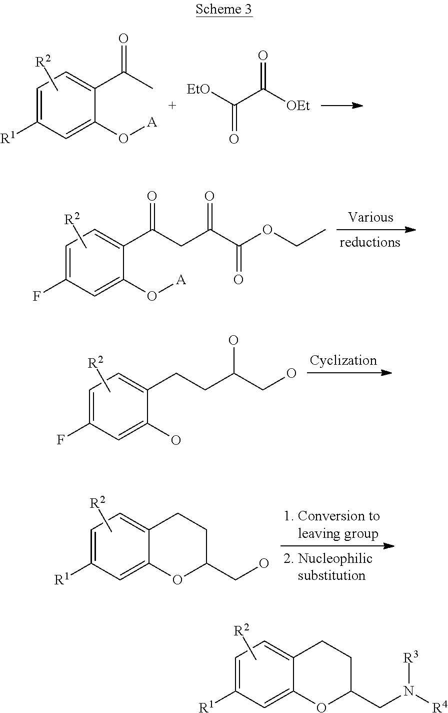

The substituents in Scheme 1-3, are as follows: z is a leaving group, G¹ is R¹ or a group that can be transformed into R¹, A is alkyl, hydrogen or a protecting group. R¹, R², R³ and R⁴ are as defined above.

### Example 1

### N-{[(2S)-7-BROMO-2,3-DIHYDRO-1,4-BENZODIOXIN-2-YL]METHYL}PROPAN-1-AMINE

A mixture of [(2R)-7-bromo-2,3-dihydro-1,4-benzodioxin-2-yl]methyl 4-methylbenzenesulfonate (3.1 g, 7.8 mmol) K₂CO₃ (1.3 g, 9.3 mmol) and propan-1-amine (0.70 ml, 8.5 mmol) in ACN (15 ml) was split into 3 batches and heated under microwave radiation to 180° C. for 10 min. After cooling to ambient temperature, the reaction mixtures were brought together and then filtered through a pad of celite and evaporated to dryness. Purification by flash column chromatography (EtOAc) gave the title compound (1.79 g, 81%). The amine was converted to the hydrochloric acid salt and crystallized from EtOH/Et₂O: M.p. 194° C. MS m/z (rel. intensity, 70 eV) 286 (M+, 4), 285 (M+, 5), 78 (13), 72 (bp), 51 (10). [α]=−75° (methanol).

### Example 2

### N-[(6,7-DIFLUORO-2,3-DIHYDRO-1,4-BENZODIOXIN-2-YL)METHYL]PROPAN-1-AMINE

A mixture of (6,7-difluoro-2,3-dihydro-1,4-benzodioxin-2-yl)methyl 4-methylbenzenesulfonate (0.44 g, 2.5 mmol), propan-1-amine (1.5 ml) and ACN (4 ml) was heated under microwave radiation to 140° C. for 20 min. Yield: 0.2 g, (32%). The amine was converted to the hydrobromic acid salt and crystallized from EtOH/diisopropyl ether: M.p. 262° C. MS m/z (rel. intensity, 70 eV) 243 (M+, 10), 117 (5), 116 (5), 88 (14), 72 (bp).

### Example 3

### N-[(7-FLUORO-2,3-DIHYDRO-1,4-BENZODIOXIN-2-YL)METHYL]PROPAN-1-AMINE

A mixture of (7-fluoro-2,3-dihydro-1,4-benzodioxin-2-yl)methyl 4-methylbenzenesulfonate (0.87 g, 2.6 mmol) and propan-1-amine (2 ml) in ACN (3 ml) was heated under microwave radiation at 130° C. for 12 min. The solution was evaporated to dryness and purified on a SCX-3 cation exchange column. Further purification on flash column chromatography (EtOAc/MeOH) gave the title product (0.32 g, 55%). The amine was converted to the hydrochloric acid salt and crystallized from EtOH/diethyl ether: M.p. 187° C. MS m/z (rel. intensity, 70 eV) 225 (M+, 23), 139 (4), 98 (8), 72 (bp), 70 (9).

### Example 4

### N-[(7-CHLORO-2,3-DIHYDRO-1,4-BENZODIOXIN-2-YL)METHYL]PROPAN-1-AMINE

A mixture of (7-chloro-2,3-dihydro-1,4-benzodioxin-2-yl)methyl 4-methylbenzenesulfonate (1.3 g, 3.7 mmol) and propan-1-amine (1 ml) in ACN (3 ml) was heated under microwave radiation to 120° C. for 20 min and then to 130° C. for 10 min. The reaction mixture was purified on a SCX-3 cation exchange column (MeOH/TEA 4:1) and filtered through a pad of silica (EtOAc/MeOH) to give the title compound (0.46 g, 52%). The amine was converted to the hydrochloric acid salt and crystallized from EtOH/Et₂O: M.p. 191° C. MS m/z (rel. intensity, 70 eV) 241 (M+, 26), 243 (M+, 8), 106 (5), 73 (5), 72 (bp).

### Example 5

### 3-[(PROPYLAMINO)METHYL]-2,3-DIHYDRO-1,4-BENZODIOXIN-6-YL METHANESULFONATE

A mixture of {7-[(methylsulfonyl)oxy]-2,3-dihydro-1,4-benzodioxin-2-yl}methyl 4-methylbenzenesulfonate (1 g, 2.4 mmol) and propan-1-amine (1 ml) in ACN (6 ml) was heated under microwave radiation to 120° C. for 20 min. The mixture was evaporated to dryness, Na₂CO₃ and EtOAc were added and the phases were separated. The combined organic phases were dried (Na₂SO₄) and evaporated to dryness. Purification on flash column chromatography gave the title compound. Yield: 0.4 g (54%). The amine was converted to the fumaric acid salt and crystallized from EtOH/Et₂O. M.p. 178° C. MS m/z (rel. intensity, 70 eV) 301 (M+, 28), 272 (5), 222 (4), 151 (5), 72 (bp).

### Example 6

### 3-[(PROPYLAMINO)METHYL]-2,3-DIHYDRO-1,4-BENZODIOXIN-6-YL TRIFLUOROMETHANESULFONATE

A mixture of (7-{[(trifluoromethyl)sulfonyl]oxy}-2,3-dihydro-1,4-benzodioxin-2-yl)methyl 4-methylbenzenesulfonate (0.5 g, 1.1 mmol) and propan-1-amine (0.4 ml, 5.4 mmol) in ACN (3 ml) was heated under microwave radiation at 120° C. for 20 min and then evaporated to dryness. Na₂CO₃ (10%) and EtOAc was added. The phases were separated and the organic phase dried (Na₂SO₄) and evaporated to dryness. The product was purified on silica and mixed with another batch of the same compound. The amine was converted to the fumaric acid salt and crystallized from EtOH/Et₂O. M.p. 163° C. MS m/z (rel. intensity, 70 eV) 355 (M+, 14), 326 (7), 222 (4), 72 (bp), 70 (6).

### Example 7

### N-[(7,8-DIFLUORO-2,3-DIHYDRO-1,4-BENZODIOXIN-2-YL)METHYL]PROPAN-1-AMINE

A mixture of (7,8-difluoro-2,3-dihydro-1,4-benzodioxin-2-yl)methyl 4-methylbenzenesulfonate (0.16 g, 0.4 mmol), propan-1-amine (3 ml) and ACN (3 ml) was heated under microwave radiation to 120° C. for 15 min. The solution was evaporated to dryness and the residue dissolved in EtOAc/Et₂O. The solution was extracted with HCl (10%) and the combined water phases were basified and extracted with EtOAc. The combined organic phases were washed with brine, dried (MgSO₄) and evaporated to dryness. Purification together with another batch of the same compound (0.22 g) on flash column chromatography (EtOAc) gave the title compound (0.2 g, 59%). The amine was converted to the hydrochloric acid salt and crystallized from EtOH/EtO: M.p. 184° C. MS m/z (rel. intensity, 70 eV) 243 (M+, 11), 88 (6), 73 (5), 72 (bp), 70 (6).

### Example 8

### N-[(5,7-DIFLUORO-2,3-DIHYDRO-1,4-BENZODIOXIN-2-YL)METHYL]PROPAN-1-AMINE

A mixture of (5,7-difluoro-2,3-dihydro-1,4-benzodioxin-2-yl)methyl 4-methylbenzenesulfonate (1 g, 2.8 mmol), propan-1-amine (3 ml) and ACN (2 ml) was heated under microwave radiation at 120° C. for 20 min and then evaporated to dryness. Et₂O and HCl (1%) was added and the phases were separated. The water phase was basified and extracted with EtOAc. The combined organic phases were dried and evaporated to dryness. Purification on flash column chromatography (isooctane/EtOAc) gave the title compound (0.4 g, 63%). The amine was converted to the hydrochloric acid salt and crystallized from EtOH/Et₂O. M.p. 157° C. MS m/z (rel. intensity, 70 eV) 243 (M+, 8), 88 (6), 73 (5), 72 (bp), 70 (6).

### Example 9

### 1-{[(2S)-7-FLUORO-2,3-DIHYDRO-1,4-BENZODIOXIN-2-YL]METHYL}PYRROLIDINE

A mixture of [(2R)-7-fluoro-2,3-dihydro-1,4-benzodioxin-2-yl]methyl 4-methylbenzenesulfonate (0.22 g, 0.65 mmol), K₂CO₃ (0.1 g, 0.71 mmol) and pyrrolidine (0.06 ml, 0.71 mmol) in acetonitrile (3 ml) was heated under microwave radiation to 180° C. for 5 min and filtered through a pad of celite. The resulting product was put together with another batch of the same product and purified on flash column chromatography (Isooctane/EtOAc/MeOH) to give the title compound (0.11 g, 34%). The amine was converted to the hydrochloric acid salt and crystallized from MeOH/Et₂O: M.p. 215° C. MS m/z (rel. intensity, 70 eV) 237 (M+, 10), 110 (4), 85 (6), 84 (bp), 70 (5).

### Example 10

### 1-[(7-FLUORO-2,3-DIHYDRO-1,4-BENZODIOXIN-2-YL)-N-METHYLMETHANAMINE

A solution of (7-fluoro-2,3-dihydro-1,4-benzodioxin-2-yl)methyl 4-methylbenzenesulfonate (0.032 g, 0.095 mmol) and methanamine (40% in H₂O, 0.5 ml) in ACN (2 ml) was heated under microwave radiation at 120° C. for 20 min. MS m/z (rel. intensity, 70 eV) 197 (M+, bp), 166 (11), 139 (17), 109 (12), 70 (27).

### Example 11

### N-[(7-FLUORO-2,3-DIHYDRO-1,4-BENZODIOXIN-2-YL)METHYL]BUTAN-1-AMINE

Preparation according to Example 10: (7-fluoro-2,3-dihydro-1,4-benzodioxin-2-yl)methyl 4-methylbenzenesulfonate (0.023 g, 0.068 mmol), butan-1-amine (0.5 ml), ACN (2 ml). MS m/z (rel. intensity, 70 eV) 239 (M+, 47), 139 (5), 98 (9), 86 (bp), 70 (10).

### Example 12

### 2-{[(7-FLUORO-2,3-DIHYDRO-1,4-BENZODIOXIN-2-YL)METHYL]AMINO}ETHANOL

Preparation according to Example 10: (7-fluoro-2,3-dihydro-1,4-benzodioxin-2-yl)methyl 4-methylbenzenesulfonate (0.026 g, 0.077 mmol), 2-aminoethanol (0.5 ml), ACN (2 ml). MS m/z (rel. intensity, 70 eV) 227 (M+, 14), 196 (7), 139 (5), 74 (bp), 56 (19).

### Example 13

### N-[(7-FLUORO-2,3-DIHYDRO-1,4-BENZODIOXIN-2-YL)METHYL]-N-PROPYLPROPAN-1-AMINE

Preparation according to Example 10: (7-fluoro-2,3-dihydro-1,4-benzodioxin-2-yl)methyl 4-methylbenzenesulfonate (0.016 g, 0.047 mmol), N-propylpropan-1-amine (0.5 ml), ACN (2 ml). MS m/z (rel. intensity, 70 eV) 267 (M+, 1), 238 (5), 115 (8), 114 (bp), 86 (10).

### Example 14

### N-ETHYL-N-[(7-FLUORO-2,3-DIHYDRO-1,4-BENZODIOXIN-2-YL)METHYL]PROPAN-1-AMINE

Preparation according to Example 10: (7-fluoro-2,3-dihydro-1,4-benzodioxin-2-yl)methyl 4-methylbenzenesulfonate (0.025 g, 0.074 mmol), N-ethylpropan-1-amine (0.5 ml), ACN (2 ml). MS m/z (rel. intensity, 70 eV) 253 (M+, 2), 101 (7), 100 (bp), 72 (9), 58 (8).

### Example 15

### 1-[(7-FLUORO-2,3-DIHYDRO-1,4-BENZODIOXIN-2-YL)METHYL]PIPERIDINE

Preparation according to Example 10: (7-fluoro-2,3-dihydro-1,4-benzodioxin-2-yl)methyl 4-methylbenzenesulfonate (0.039 g, 0.12 mmol), piperidine (0.5 ml), ACN (2 ml). MS m/z (rel. intensity, 70 eV) 251 (M+, 5), 124 (3), 99 (8), 98 (bp), 70 (4).

### Example 16

### N-[(7-FLUORO-2,3-DIHYDRO-1,4-BENZODIOXIN-2-YL)METHYL]ETHANAMINE

Preparation according to Example 10: (7-fluoro-2,3-dihydro-1,4-benzodioxin-2-yl)methyl 4-methylbenzenesulfonate (0.062 g, 0.18 mmol), ethanamine (70% in H₂O, 0.5 ml), ACN (2 ml). MS m/z (rel. intensity, 70 eV) 211 (34), 139 (4), 98 (5), 70 (7), 58 (bp).

### Example 17

### N-[(7-FLUORO-2,3-DIHYDRO-1,4-BENZODIOXIN-2-YL)METHYL]PROP-2-EN-1-AMINE

Preparation according to Example 10: (7-fluoro-2,3-dihydro-1,4-benzodioxin-2-yl)methyl 4-methylbenzenesulfonate (0.043 g, 0.13 mmol), prop-2-en-1-amine (0.5 ml), ACN (2 ml). MS m/z (rel. intensity, 70 eV) 223 (M+, 14), 139 (4), 99 (4), 71 (5), 70 (bp).

### Example 18

### 1-[(7-FLUORO-2,3-DIHYDRO-1,4-BENZODIOXIN-2-YL)-N,N-DIMETHYLMETHANAMINE

Preparation according to Example 10: (7-fluoro-2,3-dihydro-1,4-benzodioxin-2-yl)methyl 4-methylbenzenesulfonate (0.034 g, 0.10 mmol), N-methylmethanamine (40% in H₂O, 1 ml), ACN (2 ml). MS m/z (rel. intensity, 70 eV) 211 (M+, 11), 99 (3), 84 (3), 70 (5), 58 (bp).

### Example 19

### N-[(7-FLUORO-2,3-DIHYDRO-1,4-BENZODIOXIN-2-YL)METHYL]-N-METHYLPROPAN-1-AMINE

Preparation according to Example 10: (7-fluoro-2,3-dihydro-1,4-benzodioxin-2-yl)methyl 4-methylbenzenesulfonate (0.037 g, 0.11 mmol), N-methylpropan-1-amine (1 ml), ACN (2 ml). MS m/z (rel. intensity, 70 eV) 239 (M+, 5), 210 (3), 87 (6), 86 (bp), 58 (11).

### Example 20

### 1-[(7-FLUORO-2,3-DIHYDRO-1,4-BENZODIOXIN-2-YL)METHYL]AZETIDINE

A mixture of (7-fluoro-2,3-dihydro-1,4-benzodioxin-2-yl)methyl 4-methylbenzenesulfonate (0.037 g, 0.11 mmol) and azetidine (0.2 ml) in ACN (1 ml) was heated under microwave radiation at 120° C. for 30 min. MS m/z (rel. intensity, 70 eV) 223 (M+, 14), 166 (2), 99 (3), 71 (5), 70 (bp).

### Example 21

### 4-[(7-FLUORO-2,3-DIHYDRO-1,4-BENZODIOXIN-2-YL)METHYL]MORPHOLINE

Preparation according to Example 10: (7-fluoro-2,3-dihydro-1,4-benzodioxin-2-yl)methyl 4-methylbenzenesulfonate (0.089 g, 0.26 mmol), morpholine (1 ml), ACN (2 ml). MS m/z (rel. intensity, 70 eV) 253 (M+, 10), 101 (6), 100 (bp), 70 (5), 56 (5).

### Example 22

### N-[(7-FLUORO-2,3-DIHYDRO-1,4-BENZODIOXIN-2-YL)METHYL]-2-METHOXYETHANAMINE

Preparation according to Example 10: (7-fluoro-2,3-dihydro-1,4-benzodioxin-2-yl)methyl 4-methylbenzenesulfonate (0.037 g, 0.11 mmol), 2-methoxyethanamine (1 ml), ACN (2 ml). MS m/z (rel. intensity, 70 eV) 241 (M+, 39), 196 (23), 88 (bp), 70 (10), 56 (12).

### Example 23

### N-ETHYL-N-[(7-FLUORO-2,3-DIHYDRO-1,4-BENZODIOXIN-2-YL)METHYL]ETHANAMINE

Preparation according to Example 10: (7-fluoro-2,3-dihydro-1,4-benzodioxin-2-yl)methyl 4-methylbenzenesulfonate (0.021 g, 0.062 mmol), N-ethylethanamine (1 ml), ACN (2 ml). MS m/z (rel. intensity, 70 eV) 239 (M+, 2), 87 (6), 86 (bp), 72 (3), 58 (5).

### Example 24

### N-[(7-FLUORO-2,3-DIHYDRO-1,4-BENZODIOXIN-2-YL)METHYL]-N-METHYLETHANAMINE

Preparation according to Example 10: (7-fluoro-2,3-dihydro-1,4-benzodioxin-2-yl)methyl 4-methylbenzenesulfonate (0.027 g, 0.080 mmol), N-methylethanamine (1 ml), ACN (2 ml). MS m/z (rel. intensity, 70 eV) 225 (M+, 5), 98 (3), 73 (5), 72 (bp), 70 (4).

### Example 25

### 3-[(METHYLAMINO)METHYL]-2,3-DIHYDRO-1,4-BENZODIOXIN-6-YL METHANESULFONATE

A solution of {7-[(methylsulfonyl)oxy]-2,3-dihydro-1,4-benzodioxin-2-yl}methyl 4-methylbenzenesulfonate (0.1 g, 0.24 mmol), methanamine (HCl-salt, 0.2 g, 2.4 mmol) and K₂CO₃ (0.2 g) in ACN (3 ml) was heated under microwave radiation at 120° C. for 20 min. Methanamine (33% in EtOH, 1 ml) was added and the solution was further heated under microwave radiation at 120° C. for 20 min. MS m/z (rel. intensity, 70 eV) 273 (M+, bp), 230 (80), 151 (66), 79 (12), 70 (19).

### Example 26

### 3-[(ETHYLAMINO)METHYL]-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), ethanamine (HCl-salt, 0.2 g, 2.4 mmol), K₂CO₃ (0.2 g), ACN (3 ml). MS m/z (rel. intensity, 70 eV) 287 (M+, 15), 230 (4), 151 (6), 84 (5), 58 (bp).

### Example 27

### 3-[(BUTYLAMINO)METHYL]-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), butan-1-amine (0.2 ml), ACN (3 ml). MS m/z (rel. intensity, 70 eV) 315 (M+, 13), 151 (4), 112 (5), 86 (bp), 70 (7).

### Example 28

### 3-{[(2-HYDROXYETHYL)AMINO]METHYL}-2,3-DIHYDRO-1,4-BENZODIOXIN-6-YL METHANESULFONATE

A solution of {7-[(methylsulfonyl)oxy]-2,3-dihydro-1,4-benzodioxin-2-yl}methyl 4-methylbenzenesulfonate (0.1 g, 0.24 mmol) and 2-aminoethanol (0.1 ml) in ACN (3 ml) was heated under microwave radiation at 120° C. for 20 min. 2-aminoethanol (1 ml) was added and the mixture was heated for another 20 min at 120° C. under microwave radiation. MS m/z (rel. intensity, 70 eV) 303 (11), 272 (24), 151 (5), 74 (bp), 56 (14).

### Example 29

### 3-[(DIPROPYLAMINO)METHYL]-2,3-DIHYDRO-1,4-BENZODIOXIN-6-YL METHANESULFONATE

Preparation according to Example 28: {7-[(methylsulfonyl)oxy]-2,3-dihydro-1,4-benzodioxin-2-yl}methyl 4-methylbenzenesulfonate (0.1 g, 0.24 mmol), N-propylpropan-1-amine (0.3 ml+1 ml), ACN (3 ml). MS m/z (rel. intensity, 70 eV) 314 (M+, 6), 115 (8), 114 (bp), 112 (5), 86 (6).

### Example 30

### 3-{[ETHYL(PROPYL)AMINO]METHYL}-2,3-DIHYDRO-1,4-BENZODIOXIN-6-YL METHANESULFONATE

Preparation according to Example 28: {7-[(methylsulfonyl)oxy]-2,3-dihydro-1,4-benzodioxin-2-yl}methyl 4-methylbenzenesulfonate (0.1 g, 0.24 mmol), N-ethylpropan-1-amine (0.3 ml+1 ml), ACN (3 ml). MS m/z (rel. intensity, 70 eV) 329 (M+, 1), 101 (7), 100 (bp), 72 (5), 58 (6).

### Example 31

### 3-(PIPERIDIN-1-YLMETHYL)-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), piperidine (0.2 ml), ACN (3 ml). MS m/z (rel. intensity, 70 eV) 327 (M+, 3), 124 (5), 99 (7), 98 (bp), 55 (4).

### Example 32

### 3-[(DIMETHYLAMINO)METHYL]-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), N-methylmethanamine (40% in H₂O, 1 ml), ACN (3 ml). MS m/z (rel. intensity, 70 eV) 287 (M+, 8), 84 (4), 79 (3), 59 (4), 58 (bp).

### Example 33

### 3-{[METHYL(PROPYL)AMINO]METHYL}-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), N-methylpropan-1-amine (1 ml), ACN (3 ml). MS m/z (rel. intensity, 70 eV) 315 (M+, 3), 112 (6), 87 (6), 86 (bp), 84 (6), 58 (5).

### Example 34

### 3-(MORPHOLIN-4-YLMETHYL)-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), morpholine (0.2 ml), ACN (3 ml). MS m/z (rel. intensity, 70 eV) 329 (M+, 6), 126 (2), 101 (6), 100 (bp), 56 (4).

### Example 35

### 3-[(DIETHYLAMINO)METHYL]-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), N-ethylethanamine (0.2 ml), ACN (3 ml). MS m/z (rel. intensity, 70 eV) 315 (M+, 3), 112 (3), 87 (6), 86 (bp), 58 (4).

### Example 36

### 3-(PYRROLIDIN-1-YLMETHYL)-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), pyrrolidine (0.2 ml), ACN (3 ml). MS m/z (rel. intensity, 70 eV) 313 (M+, 4), 110 (5), 85 (6), 84 (bp), 55 (4).

### Example 37

### 3-[(ALLYLAMINO)METHYL]-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), prop-2-en-1-amine (0.2 ml), ACN (3 ml). MS m/z (rel. intensity, 70 eV) 299 (M+, 14), 220 (7), 151 (6), 96 (5), 70 (bp).

### Example 38

### 3-{[ETHYL(METHYL)AMINO]METHYL}-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), N-methylethanamine (0.2 ml), ACN (3 ml). MS m/z (rel. intensity, 70 eV) 301 (M+, 4), 98 (3), 84 (3), 73 (5), 72 (bp).

### Example 39

### 3-{[(2-METHOXYETHYL)AMINO]METHYL}-2,3-DIHYDRO-1,4-BENZODIOXIN-6-YL METHANESULFONATE

Preparation according to Example 10: {7-[(methylsulfonyl)oxy]-2,3-dihydro-1,4-benzodioxin-2-yl}methyl 4-methylbenzenesulfonate (0.1 g, 0.24 mmol), 2-methoxyethanamine (0.2 ml), ACN (3 ml). MS m/z (rel. intensity, 70 eV) 317 (M+, 12), 272 (29), 88 (bp), 70 (9), 56 (9).

### Example 40

### 3-(AZETIDIN-1-YLMETHYL)-2,3-DIHYDRO-1,4-BENZODIOXIN-6-YL METHANESULFONATE

Preparation according to Example 20: {7-[(methylsulfonyl)oxy]-2,3-dihydro-1,4-benzodioxin-2-yl}methyl 4-methylbenzenesulfonate (0.05 g, 0.012 mmol), azetidine (0.1 ml), ACN (1.5 ml). MS m/z (rel. intensity, 70 eV) 300 (M+, 9), 299 (M+, 59), 220 (6), 192 (8), 70 (bp).

### Example 41

### 3-[(METHYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), methanamine (40% in H₂O, 1 ml), ACN (3 ml). MS m/z (rel. intensity, 70 eV) 327 (M+, bp), 163 (17), 123 (15), 70 (48), 69 (68).

### Example 42

### 3-[(ETHYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), ethanamine (70% in H₂O, 1 ml), ACN (3 ml). MS m/z (rel. intensity, 70 eV) 341 (M+, 7), 84 (5), 69 (7), 59 (4), 58 (bp).

### Example 43

### 3-[(BUTYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), butan-1-amine (1 ml), ACN (3 ml). MS m/z (rel. intensity, 70 eV) 369 (M+, 5), 87 (6), 86 (bp), 70 (8), 69 (6).

### Example 44

### 3-{[(2-HYDROXYETHYL)AMINO]METHYL}-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), 2-aminoethanol (1 ml), ACN (3 ml). MS m/z (rel. intensity, 70 eV) 357 (M+, 8), 326 (19), 74 (bp), 69 (9), 56 (12).

### Example 45

### 3-[(DIPROPYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-propylpropan-1-amine (1 ml), ACN (3 ml). MS m/z (rel. intensity, 70 eV) 396 (M+, 1), 368 (41), 115 (41), 114 (bp), 112 (32).

### Example 46

### 3-{[ETHYL(PROPYL)AMINO]METHYL}-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-ethylpropan-1-amine (1 ml), ACN (3 ml). MS m/z (rel. intensity, 70 eV) 382 (M+, 1), 354 (11), 221 (6), 101 (7), 100 (bp).

### Example 47

### 3-(PIPERIDIN-1-YLMETHYL)-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), piperidine (1 ml), ACN (3 ml). MS m/z (rel. intensity, 70 eV) 381 (M+, 4), 248 (9), 124 (5), 99 (6), 98 (bp).

### Example 48

### 3-[(DIMETHYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-methylmethanamine (40% in H₂O, 1 ml), ACN (3 ml). MS m/z (rel. intensity, 70 eV) 341 (M+, 2), 84 (5), 69 (5), 59 (4), 58 (bp).

### Example 49

### 3-(MORPHOLIN-4-YLMETHYL)-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), morpholine (1 ml), ACN (3 ml). MS m/z (rel. intensity, 70 eV) 383 (M+, 1), 101 (6), 100 (bp), 70 (5), 56 (5).

### Example 50

### 3-[(DIETHYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-ethylethanamine (1 ml), ACN (3 ml). MS m/z (rel. intensity, 70 eV) 368 (M+, 1), 112 (4), 87 (5), 86 (bp), 58 (4).

### Example 51

### 3-(PYRROLIDIN-1-YLMETHYL)-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), pyrrolidine (1 ml), ACN (3 ml). MS m/z (rel. intensity, 70 eV) 367 (M+, 1), 110 (5), 85 (6), 84 (bp), 69 (4).

### Example 52

### 3-[(ALLYLAMINO)METHYL]-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), prop-2-en-1-amine (1 ml), ACN (3 ml). MS m/z (rel. intensity, 70 eV) 353 (M+, 5), 220 (3), 96 (5), 70 (bp), 69 (7).

### Example 53

### 3-{[ETHYL(METHYL)AMINO]METHYL}-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-methylethanamine (1 ml), ACN (3 ml). MS m/z (rel. intensity, 70 eV) 355 (M+, 1), 98 (4), 73 (5), 72 (bp), 69 (5).

### Example 54

### 3-{[(2-METHOXYETHYL)AMINO]METHYL}-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), 2-methoxyethanamine (1 ml), ACN (3 ml). MS m/z (rel. intensity, 70 eV) 371 (M+, 2), 326 (17), 88 (bp), 70 (17), 56 (9).

### Example 55

### 3-(AZETIDIN-1-YLMETHYL)-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.05 g, 0.1 mmol), azetidine (0.1 ml), ACN (1.5 ml). MS m/z (rel. intensity, 70 eV) 353 (M+, 1), 192 (2), 71 (5), 70 (bp), 69 (6).

### Example 56

### 3-{[METHYL(PROPYL)AMINO]METHYL}-2,3-DIHYDRO-1,4-BENZODIOXIN-6-YL TRIFLUOROMETHANESULFONATE

Preparation according to Example 10: (7-{[(trifluoromethyl)sulfonyl]oxy}-2,3-dihydro-1,4-benzodioxin-2-yl)methyl 4-methylbenzenesulfonate (0.1 g, 0.21 mmol), N-methylpropan-1-amine (1 ml), ACN (3 ml). MS m/z (rel. intensity, 70 eV) 369 (M+, 1), 340 (11), 207 (7), 86 (bp), 84 (7).

### Example 57

### 1-[(6,7-DIFLUORO-2,3-DIHYDRO-1,4-BENZODIOXIN-2-YL)METHYL]PYRROLIDINE

A mixture of (6,7-difluoro-2,3-dihydro-1,4-benzodioxin-2-yl)methyl 4-methylbenzenesulfonate (0.44 g, 1.2 mmol) and pyrrolidin (1.5 ml) in ACN (4 ml) was heated under microwave radiation at 140° C. for 15 min and then mixed with another batch of the same product (0.4 g sm). The combined batches were evaporated to dryness and dissolved in EtOAc and H₂O. The organic phase was washed with H₂O and extracted with HCl (10%). The waterphase was basified (Na₂CO₃) and extracted with EtOAc. Yield: (0.2 g, 40%). The amine was converted to the hydrobromic acid salt and crystallized from EtOH/diisopropylether. MS m/z (rel. intensity, 70 eV) 255 (M+, 2), 88 (6), 85 (6), 84 (bp), 55 (7).

### Example 58

### N-{[(2S)-7-(TRIFLUOROMETHYL)-2,3-DIHYDRO-1,4-BENZODIOXIN-2-YL]METHYL}PROPAN-1-AMINE

A mixture of [(2R)-7-(trifluoromethyl)-2,3-dihydro-1,4-benzodioxin-2-yl]methyl 4-methylbenzenesulfonate (0.48 g, 1.23 mmol), propan-1-amine (1 ml) and ACN (2 ml) was heated under microwave radiation at 120° C. for 30 min. Propanamine (0.5 ml) was added and the mixture was heated under microwave radiation again at 120° C. for 30 min Purification on SCX-3 column (TEA/MeOH) and by preparative HPLC (MeOH/NH₃ buffer). Yield: 0.12 g, 40%. The amine was converted to the hydrochloric acid salt and crystallized from MeOH/Et₂O. M.p. 166° C. MS m/z (rel. intensity, 70 eV) 275 (M+, 3), 120 (4), 73 (5), 72 (bp), 70 (6). [α]=−52° (methanol).

### Example 59

### 1-(5,7-DIFLUORO-2,3-DIHYDRO-1,4-BENZODIOXIN-2-YL)-N-METHYLMETHANAMINE

Preparation according to Example 10: (5,7-difluoro-2,3-dihydro-1,4-benzodioxin-2-yl)methyl 4-methylbenzenesulfonate (0.020 g, 0.0561 mmol), methanamine (33% in EtOH, 0.5 ml), ACN (3 ml). MS m/z (rel. intensity, 70 eV) 215 (M+, bp), 145 (29), 117 (48), 88 (95), 70 (55).

### Example 60

### N-[(5,7-DIFLUORO-2,3-DIHYDRO-1,4-BENZODIOXIN-2-YL)METHYL]ETHANAMINE

Preparation according to Example 10: (5,7-difluoro-2,3-dihydro-1,4-benzodioxin-2-yl)methyl 4-methylbenzenesulfonate (0.020 g, 0.0561 mmol), ethanamine (2.0 M in MeOH, 0.5 ml), ACN (3 ml). MS m/z (rel. intensity, 70 eV) 229 (M+, 10), 87 (11), 70 (5), 58 (bp), 56 (5).

### Example 61

### N-[(5,7-DIFLUORO-2,3-DIHYDRO-1,4-BENZODIOXIN-2-YL)METHYL]PROP-2-EN-1-AMINE

Preparation according to Example 10: (5,7-difluoro-2,3-dihydro-1,4-benzodioxin-2-yl)methyl 4-methylbenzenesulfonate (0.020 g, 0.0561 mmol), prop-2-en-1-amine (0.5 ml), ACN (3 ml). MS m/z (rel. intensity, 70 eV) 241 (M+, 5), 117 (6), 88 (9), 70 (bp), 68 (7).

### Example 62

### 4-[(5,7-DIFLUORO-2,3-DIHYDRO-1,4-BENZODIOXIN-2-YL)METHYL]MORPHOLINE

Preparation according to Example 10: (5,7-difluoro-2,3-dihydro-1,4-benzodioxin-2-yl)methyl 4-methylbenzenesulfonate (0.020 g, 0.0561 mmol), morpholine (0.5 ml), ACN (3 ml). MS m/z (rel. intensity, 70 eV) 271 (M+, 3), 101 (7), 100 (bp), 88 (5), 70 (4), 56 (8).

### Example 63

### N-[(5,7-DIFLUORO-2,3-DIHYDRO-1,4-BENZODIOXIN-2-YL)METHYL]BUTAN-1-AMINE

Preparation according to Example 10: (5,7-difluoro-2,3-dihydro-1,4-benzodioxin-2-yl)methyl 4-methylbenzenesulfonate (0.020 g, 0.0561 mmol), butan-1-amine (0.5 ml), ACN (3 ml). MS m/z (rel. intensity, 70 eV) 257 (M+, 11), 88 (14), 86 (bp), 70 (11), 57 (8).

### Example 64

### N-[(5,7-DIFLUORO-2,3-DIHYDRO-1,4-BENZODIOXIN-2-YL)METHYL]-N-PROPYLPROPAN-1-AMINE

Preparation according to Example 10: (5,7-difluoro-2,3-dihydro-1,4-benzodioxin-2-yl)methyl 4-methylbenzenesulfonate (0.020 g, 0.0561 mmol), N-propylpropan-1-amine (0.5 ml), ACN (3 ml). MS m/z (rel. intensity, 70 eV) 285 (M+, 1), 256 (10), 115 (8), 114 (bp), 86 (12), 72 (6).

### Example 65

### 1-(5,7-DIFLUORO-2,3-DIHYDRO-1,4-BENZODIOXIN-2-YL)-N,N-DIMETHYLMETHANAMINE

Preparation according to Example 10: (5,7-difluoro-2,3-dihydro-1,4-benzodioxin-2-yl)methyl 4-methylbenzenesulfonate (0.020 g, 0.0561 mmol), N-methylmethanamine (2.0 M in MeOH, 0.5 ml), ACN (3 ml). MS m/z (rel. intensity, 70 eV) 229 (M+, 2), 117 (3), 88 (7), 84 (3), 59 (4), 58 (bp).

### Example 66

### N-[(5,7-DIFLUORO-2,3-DIHYDRO-1,4-BENZODIOXIN-2-YL)METHYL]-N-ETHYLETHANAMINE

Preparation according to Example 10: (5,7-difluoro-2,3-dihydro-1,4-benzodioxin-2-yl)methyl 4-methylbenzenesulfonate (0.020 g, 0.0561 mmol), N-ethylethanamine (0.5 ml), ACN (3 ml). MS m/z (rel. intensity, 70 eV) 257 (M+, 1), 88 (5), 87 (7), 86 (100), 58 (8), 56 (5).

### Example 67

### N-[(5,7-DIFLUORO-2,3-DIHYDRO-1,4-BENZODIOXIN-2-YL)METHYL]PROPAN-2-AMINE

Preparation according to Example 10: (5,7-difluoro-2,3-dihydro-1,4-benzodioxin-2-yl)methyl 4-methylbenzenesulfonate (0.020 g, 0.0561 mmol), propan-2-amine (0.5 ml), ACN (3 ml). MS m/z (rel. intensity, 70 eV) 243 (M+, 9), 88 (16), 84 (13), 72 (bp), 56 (10).

### Example 68

### N-[(5,7-DIFLUORO-2,3-DIHYDRO-1,4-BENZODIOXIN-2-YL)METHYL]-N-METHYLPROPAN-1-AMINE

Preparation according to Example 10: (5,7-difluoro-2,3-dihydro-1,4-benzodioxin-2-yl)methyl 4-methylbenzenesulfonate (0.020 g, 0.0561 mmol), N-methylpropan-1-amine (N—,N—) (0.5 ml), ACN (3 ml). MS m/z (rel. intensity, 70 eV) 257 (M+, 1), 88 (9), 87 (6), 86 (bp), 84 (9), 58 (16).

### Example 69

### N-[(5,7-DIFLUORO-2,3-DIHYDRO-1,4-BENZODIOXIN-2-YL)METHYL]-N-ETHYLPROPAN-1-AMINE

Preparation according to Example 10: (5,7-difluoro-2,3-dihydro-1,4-benzodioxin-2-yl)methyl 4-methylbenzenesulfonate (0.020 g, 0.0561 mmol), N-ethylpropan-1-amine (0.5 ml), ACN (3 ml). MS m/z (rel. intensity, 70 eV) 271 (M+, 1), 101 (8), 100 (bp), 98 (6), 72 (13), 58 (13).

### Example 70

### 2-{[(5,7-DIFLUORO-2,3-DIHYDRO-1,4-BENZODIOXIN-2-YL)METHYL]AMINO}ETHANOL

Preparation according to Example 10: (5,7-difluoro-2,3-dihydro-1,4-benzodioxin-2-yl)methyl 4-methylbenzenesulfonate (0.020 g, 0.0561 mmol), 2-aminoethanol (0.5 ml), ACN (3 ml). ¹H-NMR (400 MHz, CDCl₃): δ 6.43-6.49 (2H, m), δ 4.30-4.34 (2H, m), δ 4.04 (1H, dd, J=11.6 Hz, J 7.6), 3.68 (2H, t, J=5.2 Hz), δ 2.93 (2H, t, J=4.8 Hz), δ 2.84 (2H, t, J=4.8 Hz).

### Example 71

### N-[(5,7-DIFLUORO-2,3-DIHYDRO-1,4-BENZODIOXIN-2-YL)METHYL]-N-METHYLETHANAMINE

Preparation according to Example 10: (5,7-difluoro-2,3-dihydro-1,4-benzodioxin-2-yl)methyl 4-methylbenzenesulfonate (0.020 g, 0.0561 mmol), N-methylethanamine (0.5 ml), ACN (3 ml). MS m/z (rel. intensity, 70 eV) 243 (M+, 1), 117 (3), 88 (7), 84 (4), 73 (5), 72 (bp).

### Example 72

### N-[(5,7-DIFLUORO-2,3-DIHYDRO-1,4-BENZODIOXIN-2-YL)METHYL]-2-METHOXYETHANAMINE

Preparation according to Example 10: (5,7-difluoro-2,3-dihydro-1,4-benzodioxin-2-yl)methyl 4-methylbenzenesulfonate (0.020 g, 0.0561 mmol), 2-methoxyethanamine (0.5 ml), ACN (3 ml). MS m/z (rel. intensity, 70 eV) 259 (M+, 9), 214 (13), 88 (bp), 70 (11), 58 (10), 56 (19).

### Example 73

### 1-[(5,7-DIFLUORO-2,3-DIHYDRO-1,4-BENZODIOXIN-2-YL)METHYL]AZETIDINE

Preparation according to Example 10: (5,7-difluoro-2,3-dihydro-1,4-benzodioxin-2-yl)methyl 4-methylbenzenesulfonate (0.020 g, 0.0561 mmol), azetidine (0.2 ml), ACN (3 ml). MS m/z (rel. intensity, 70 eV) 241 (M+, 6), 116 (5), 88 (9), 71 (5), 70 (bp).

### Example 74

### N-[(5,7-DIFLUORO-2,3-DIHYDRO-1,4-BENZODIOXIN-2-YL)METHYL]-2-METHYLPROPAN-1-AMINE

Preparation according to Example 10: (5,7-difluoro-2,3-dihydro-1,4-benzodioxin-2-yl)methyl 4-methylbenzenesulfonate (0.020 g, 0.0561 mmol), 2-methylpropan-1-amine (0.5 ml), ACN (3 ml). MS m/z (rel. intensity, 70 eV) 257 (M+, 11), 214 (14), 88(14), 86 (bp), 70 (12), 57 (15).

### Example 75

### 1-[(5,7-DIFLUORO-2,3-DIHYDRO-1,4-BENZODIOXIN-2-YL)METHYL]PYRROLIDINE

Preparation according to Example 10: (5,7-difluoro-2,3-dihydro-1,4-benzodioxin-2-yl)methyl 4-methylbenzenesulfonate (0.020 g, 0.0561 mmol), pyrrolidine (0.5 ml), ACN (3 ml). MS m/z (rel. intensity, 70 eV) 255 (M+, 2), 110 (5), 88 (6), 84 (bp), 85 (6), 55 (5).

### Example 76

### 1-[(5,7-DIFLUORO-2,3-DIHYDRO-1,4-BENZODIOXIN-2-YL)METHYL]PIPERIDINE

Preparation according to Example 10: (5,7-difluoro-2,3-dihydro-1,4-benzodioxin-2-yl)methyl 4-methylbenzenesulfonate (0.020 g, 0.0561 mmol), piperidine (0.5 ml), ACN (3 ml). MS m/z (rel. intensity, 70 eV) 269 (M+, 1), 99 (7), 98 (bp), 88 (5), 70 (4), 55 (6).

### Example 77

### N-[(5,7-DIFLUORO-2,3-DIHYDRO-1,4-BENZODIOXIN-2-YL)METHYL]-3-FLUOROPROPAN-1-AMINE

A solution of (5,7-difluoro-2,3-dihydro-1,4-benzodioxin-2-yl)methyl 4-methylbenzenesulfonate (0.020 g, 0.0561 mmol), 3-fluoropropan-1-amine (0.23 M in MeOH 80%/TEA 20%, 1 ml), ACN (3 ml) was heated under microwave radiation at 120° C. for 1 h 20 min. MS m/z (rel. intensity, 70 eV) 261 (M+, 6), 91(5), 90 (bp), 88 (12), 70 (10).

### Example 78

### 2-({[(2S)-7-(TRIFLUOROMETHYL)-2,3-DIHYDRO-1,4-BENZODIOXIN-2-YL]METHYL}AMINO)ETHANOL

A mixture of [(2R)-7-(trifluoromethyl)-2,3-dihydro-1,4-benzodioxin-2-yl]methyl 4-methylbenzenesulfonate (0.25 g, 0.64 mmol), 2-aminoethanol (1 ml) and ACN (2 ml) was heated under microwave radiation at 120° C. for 30 min. Purification by preparative HPLC (MeOH/NH₃ buffer). Yield: 0.060 g, 34%. The amine was converted to the oxalic acid salt and crystallized from EtOH. M.p. 181.5-181.9° C. ¹H-NMR (400 MHz, CD₃OD): δ 7.17 (1H, d, J 2.3), δ 7.13 (1H, dd, J 8.6, 2.3), δ 7.02 (1H, d, J8.69), δ 4.39 (1H, dd, J 11.5, 2.3), δ 4.34 (1H, m), δ 4.05 (1H, dd J 11.5, 7.2), δ 3.68 (2H, t, J 5.5), δ 2.91 (2H, dd, J 7.0, 5.0), δ 2.79 (2H, dd, J 7.0, 5.0) ppm (J-values are in Hz and shifts relative to solvent-peak at 3.31 ppm) [α]=−43° (methanol).

### Example 79

### N-{[7-(FLUOROMETHYLSULFONYL)-3,4-DIHYDRO-2H-CHROMEN-2-YL]METHYL}-N-PROPAN-1-AMINE

A mixture of (7-fluoro-3,4-dihydro-2h-chromen-2-yl)methyl 4-methylbenzenesulfonate (0.58 g, 1.5 mmol), propan-1-amine (2 ml) and acetonitrile (8 ml) was heated under microwave radiation at 120° C. for 30 min. The product was evaporated to dryness and was dissolved in EtOAc and extracted with HCl (10% in H₂O). The combined water phases were basified using NaOH (20%). Extraction of the water phase using EtOAc gave the title compound (0.33 g, 99%). The amine was converted to the hydrochloric acid salt and recrystallized from MeOH. M.p. 212.8-213.6° C. MS m/z (rel. intensity, 70 eV) 223 (M+, 28), 194 (13), 125 (7), 96 (8), 72 (bp).

### PREPARATIONS

### Preparation 1

### 5-BROMO-2-[(2S)-OXIRAN-2-YLMETHOXY]BENZALDEHYDE

A mixture of 5-bromo-2-hydroxybenzaldehyde (10 g, 49.8 mmol), R-glycidyltosylate (11.4 g, 49.8 mmol) and K₂CO₃ (6.9 g, 49.8 mmol) in DMF (120 ml) was heated at 100° C. for 4 h. The mixture was cooled to ambient temperature, water and EtOAc were added. The phases were separated and the combined organic phases were washed with brine, dried (MgSO₄), filtered and concentrated to give the title compound (10.9 g). MS m/z (rel. intensity, 70 eV) 258 (M+, 88), 256 (M+, 82), 200 (89), 198 (98), 63 (99), 57 (bp).

### Preparation 2

### 5-BROMO-2-[(2S)-OXIRAN-2-YLMETHOXY]PHENYL FORMATE

To a solution of 5-bromo-2-[(2S)-oxiran-2-ylmethoxy]benzaldehyde (10.9 g, 42.2 mmol) in DCM (200 ml) was added m-CPBA (77%, 13.2 g, 59.1 mmol). The solution was heated at reflux over night and then brought to ambient temperature. Aqueous Na₂CO₃ (10%) was added and the phases were separated. The organic phase was washed with brine, dried (MgSO₄), filtered and evaporated to dryness. The residue was purified by flash column chromatography (isooctane/EtOAc) to give the title compound (9.02 g). MS m/z (rel. intensity, 70 eV) 275 (M+, 4), 273 (M+, 4), 189 (46), 187 (48), 57 (bp), 51 (79).

### Preparation 3

### [(2S)-7-BROMO-2,3-DIHYDRO-1,4-BENZODIOXIN-2-YL]METHANOL

A mixture of 5-bromo-2-[(2S)-oxiran-2-ylmethoxy]phenyl formate (9 g, 33.0 mmol) and NaOH (2 M, 50 ml) was heated at reflux for 1 h 30 min and left at room temperature over night. The mixture was extracted with EtOAc. The combined organic phases were washed with water and dried (MgSO₄). The oily product was purified by flash column chromatography (isooctane/EtOAc) to give the title compound (4.9 g). MS m/z (rel. intensity, 70 eV) 245 (M+, 93) 244 (M+, bp), 188 (62), 79 (88), 51 (69).

### Preparation 4

### [(2R)-7-BROMO-2,3-DIHYDRO-1,4-BENZODIOXIN-2-YL]METHYL 4-METHYLBENZENESULFONATE

To a solution of [(2S)-7-bromo-2,3-dihydro-1,4-benzodioxin-2-yl]methanol (4.9 g, 20.0 mmol) in pyridine (10 ml) was added toluenesulfonyl chloride (6.7 g, 22.0 mmol). The mixture was split into six equal batches that were heated under microwave radiation to 100° C. for 60 sek. The batches were mixed, HCl (10% in H₂O) and EtOAc was added and the phases were separated. The combined organic phases were washed with brine, dried (MgSO₄) and evaporated to dryness. The residue was purified by flash column chromatography to give the title compound (5.7 g). MS m/z (rel. intensity, 70 eV) 400 (M+, 44), 399 (M+, 41), 227 (50), 226 (52), 91 (bp).

### Preparation 5

### 4,5-DIFLUORO-2-METHOXYPHENOL

To a solution of 1,2-difluoro-4,5-dimethoxybenzene (5 g, 28.7 mmol) in DMF (14 ml) was added sodium thiomethoxide (6.2 g, 88.5 mmol). After 30 min more DMF (4 ml) was added. The mixture was stirred in room temperature for 1 h 30 min and cooled on an icebath. Water was added and then HCl (10% in H₂O). The solution was extracted with EtOAc and the combined organic phases were washed with water, dried (MgSO₄) and evaporated to dryness with EtOH. Purification on flash column chromatography (Isooctane/EtOAc) gave the title compound (1.5 g). MS m/z (rel. intensity, 70 eV) 160 (M+, 82) 145 (bp), 117 (63), 97 (8), 88 (9).

### Preparation 6

### 2-[(4,5-DIFLUORO-2-METHOXYPHENOXY)METHYL]OXIRANE

4,5-difluoro-2-methoxyphenol (0.4 g, 2.6 mmol) was dissolved in EtOH (20 ml). Epibromhydrin (0.3 ml, 3.4 mmol) was added, followed by KOH (0.2 g, 2.8 mmol) and water (0.5 ml). The resulting solution was heated at reflux for 1 h 45 min. HCl (10% in H₂O) was added and the mixture was evaporated. Water and EtOAc was added and the phases were separated. The organic phase was evaporated to dryness with dry EtOH to give the title compound in a mixture with sideproducts that were not separated. Yield: 0.7 g. MS m/z (rel. intensity, 70 eV) 216 (M+, 78), 160 (77), 145 (bp), 88 (71), 57 (59).

### Preparation 7

### 4,5-DIFLUORO-2-(OXIRAN-2-YLMETHOXY)PHENOL

2-[(4,5-difluoro-2-methoxyphenoxy)methyl]oxirane (0.8 g, 3.6 mmol) was cooled on an icebath and HBr (48%, 10 ml) was added slowly. The reaction mixture was heated at 105° C. for 3 h and then brought to ambient temperature. Water and EtOAc was added and phases were separated. The combined organic phases were evaporated to dryness with dry EtOH to give the title compound in a mixture with sideproducts that were not separated. MS m/z (rel. intensity, 70 eV) 202 (M+, 68), 146 (bp), 117 (61), 88 (40), 57 (62).

### Preparation 8

### (6,7-DIFLUORO-2,3-DIHYDRO-1,4-BENZODIOXIN-2-YL)METHANOL

A mixture of 4,5-difluoro-2-(oxiran-2-ylmethoxy)phenol (0.5 g, 2.6 mmol) and KOH (0.2 g, 2.8 mmol) in EtOH (50 ml) was heated at reflux for 3 h. Another (0.2 g, 2.2 mmol) of KOH was added and the mixture was heated at reflux for 2 h 30 min and then brought to ambient temperature. HCl (10% in H₂O, 2 ml) and water was added and the solution was stirred over night and then evaporated to dryness with dry EtOH. The residue was purified on flash column chromatography (Isooctane/EtOAc/MeOH) to give the title compound (0.1 g). MS m/z (rel. intensity, 70 eV) 202 (M+, 86), 146 (bp), 145 (38), 88 (49), 57 (45).

### Preparation 9

### (6,7-DIFLUORO-2,3-DIHYDRO-1,4-BENZODIOXIN-2-YL)METHYL 4-METHYLBENZENESULFONATE

(6,7-difluoro-2,3-dihydro-1,4-benzodioxin-2-yl)methanol (0.8 g, 3.5 mmol), TEA (0.8 ml, 5.9 mmol) and p-toluenesulfonyl chloride (1.1 g, 5.9 mmol) was dissolved in DCM. The resulting solution was stirred in room temperature over night. H₂O and HCl (10% in H₂O) was added and the phases were separated. The organic phase was washed with Na₂CO₃ (10% in H₂O) and evaporated to dryness with dry EtOH to give the title compound. Yield: 1.8 g. MS m/z (rel. intensity, 70 eV) 356 (M+, 55), 184 (bp), 183 (40), 145 (28), 91 (69).

### Preparation 10

### 5-FLUORO-2-(OXIRAN-2-YLMETHOXY)BENZALDEHYDE

A mixture of 5-fluoro-2-hydroxybenzaldehyde (10 g, 35.7 mmol), epibromhydrin (5.8 ml, 35.7 mmol) and K₂CO₃ (9.8 g, 35.7 mmol) in DMF was heated at 100° C. for 20 min. After cooling to ambient temperature water and EtOAc were added. The phases were separated and the combined organic phases washed with LiCl (5% in H₂O, 100 ml), dried (MgSO₄) and evaporated to dryness. Purification by flash column chromatography (isooctane/EtOAc) gave the title compound (8.8 g). MS m/z (rel. intensity, 70 eV) 196 (M+, 28), 139 (bp), 138 (65), 83 (38), 57 (54).

### Preparation 11

### 5-FLUORO-2-(OXIRAN-2-YLMETHOXY)PHENOL

m-CPBA (77%, 4.6 g, 20.7 mmol) was slowly added to a solution of 5-fluoro-2-(oxiran-2-ylmethoxy)benzaldehyde (2.9 g, 14.8 mmol) in DCM (20 ml). The mixture was heated at reflux for 3 h and then brought to ambient temperature. Aqueous Na₂CO₃ (10%) and EtOAc was added and the phases were separated. The combined organic phases were dried (MgSO₄) and purified with flash column chromatography to give the title compound (1.4 g). MS m/z (rel. intensity, 70 eV) 184 (M+, bp), 138 (32), 128 (88), 99 (40), 57 (32).

### Preparation 12

### (7-FLUORO-2,3-DIHYDRO-1,4-BENZODIOXIN-2-YL)METHANOL

5-fluoro-2-(oxiran-2-ylmethoxy)phenol (1.8 g, 9.8 mmol) in sodium hydroxide (15% in H₂O, 15 ml) was heated at reflux for 1 h. The mixture was cooled to ambient temperature and extracted with diethyl ether. The combined organic phases were washed with water, dried (MgSO₄) and evaporated to dryness to give the title compound (2.2 g). MS m/z (rel. intensity, 70 eV) 184 (M+, bp), 153 (34), 138 (26), 128 (70), 127 (20).

### Preparation 13

### (7-FLUORO-2,3-DIHYDRO-1,4-BENZODIOXIN-2-YL)METHYL 4-METHYLBENZENESULFONATE

A mixture of (7-fluoro-2,3-dihydro-1,4-benzodioxin-2-yl)methanol (0.92 g, 5 mmol) p-toluenesulfonyl chloride (1.43 g, 7.5 mmol), TEA (1.04 ml, 7.5 mmol) and 4-DMAP (0.61 g, 5 mmol) in DCM was stirred in room temperature for 1 h. Water and EtOAc was added and the combined organic phases were dried (MgSO₄) and evaporated to dryness to give the title compound (1.19 g). MS m/z (rel. intensity, 70 eV) 338 (M+, bp), 166 (59), 165 (37), 139 (17), 91 (23).

### Preparation 14

### 5-CHLORO-2-(OXIRAN-2-YLMETHOXY)BENZALDEHYDE

To a solution of 5-chloro-2-hydroxybenzaldehyde (8 g, 51.2 mmol) in DMF (30 ml) was added epibromhydrin (4.2 ml, 51.2 mmol) and K₂CO₃ (7.1 g, 51.2 mmol) and the mixture was heated at 100° C. for 1 h. The mixture was cooled to ambient temperature and water and EtOAc was added. The phases were separated, and the combined organic phases washed with aqueous LiCl (10% in H₂O), dried (MgSO₄) and evaporated to give the title product (15 g including impurities of DMF). MS m/z (rel. intensity, 70 eV) 212 (M+, 54), 169 (32), 156 (65), 155 (bp), 156 (64).

### Preparation 15

### 5-CHLORO-2-(OXIRAN-2-YLMETHOXY)PHENOL

To a solution of 5-chloro-2-(oxiran-2-ylmethoxy)benzaldehyde [15 g (including impurities of DMF), 51.2 mmol] in DCM (40 ml) was added m-CPBA (77%, 16.1 g, 71.7 mmol) and the mixture was heated at reflux for 1 h and then brought to ambient temperature. The solution was washed with Na₂CO₃ (10% in H₂O) and extracted with EtOAc. The combined organic phases were dried (MgSO₄) and evaporated to dryness to give the title product in a mixture with sideproducts that were not separated (9.39 g). LC-MS m/z (ESI) 199 (M+1).

### Preparation 16

### (7-CHLORO-2,3-DIHYDRO-1,4-BENZODIOXIN-2-YL)METHANOL

A mixture of 5-chloro-2-(oxiran-2-ylmethoxy)phenol (9.39 g, 41.4 mmol) and sodium hydroxide (15% in H₂O, 30 ml) was heated at reflux for 30 min. The mixture was cooled to ambient temperature and extracted with Et₂O and EtOAc. The combined organic phases were dried (MgSO₄), evaporated to dryness and purified on flash column chromatography (EtOAc/MeOH) to give the title compound (2.7 g). MS m/z (rel. intensity, 70 eV) 200 (M+, bp), 169 (40), 144 (83), 79 (42), 51 (46).

### Preparation 17

### (7-CHLORO-2,3-DIHYDRO-1,4-BENZODIOXIN-2-YL)METHYL 4-METHYLBENZENESULFONATE

To a solution of (7-chloro-2,3-dihydro-1,4-benzodioxin-2-yl)methanol (2.7 g, 13.5 mmol) in DCM (30 ml) was added p-toluenesulfonyl chloride (3.9 g, 20.3 mmol), TEA (2.8 ml, 20.3 mmol) and 4-DMAP (1.65 g, 13.5 mmol). The mixture was stirred at ambient temperature for 45 min, water and EtOAc were added and the phases were separated. The combined organic phases were dried (MgSO₄) and evaporated to dryness. Purification on flash column chromatography (MeOH) gave the title compound (1.3 g). MS m/z (rel. intensity, 70 eV) 354 (M+, bp) 356 (M+, 38), 182 (92), 181 (45), 91 (50).

### Preparation 18

### 1-[5-METHOXY-2-(OXIRAN-2-YLMETHOXY)PHENYL]ETHANONE

To a mixture of 1-(2-hydroxy-5-methoxyphenyl)ethanone (10 g, 60 mmol) and K₂CO₃ (12.4 g) in DMF (50 ml) was added epibromhydrin (15 ml). The solution was heated at 80° C. and the reaction followed with GC/MS. H₂O and EtOAc was added and the resulting solution was extracted several times with EtOAc. The combined organic layers were washed with aqueous LiCl (5%), HCl (1N) and brine. Drying (Na₂SO₄) and evaporation of the solvent gave the title compound. Yield: 15 g (with impurities of DMF). MS m/z (rel. intensity, 70 eV) 222 (M+, 73), 180 (26), 166 (52), 151 (bp), 137 (27).

### Preparation 19

### 5-METHOXY-2-(OXIRAN-2-YLMETHOXY)PHENYL ACETATE

To a solution of 1-[5-methoxy-2-(oxiran-2-ylmethoxy)phenyl]ethanone (13.3 g, 60 mmol) in DCM (100 ml) was slowly added m-CPBA (77%, 20.7 g, 50 mmol). The mixture was heated at reflux for 18 h and then brought to ambient temperature. The solution was washed with aqueous NaHCO₃ and with brine. The organic phase was dried (Na₂SO₄) and evaporated to dryness to give the crude title compound that was used directly in the next step (Preparation 20) without further analysis.

### Preparation 20

### (7-METHOXY-2,3-DIHYDRO-1,4-BENZODIOXIN-2-YL)METHANOL

5-methoxy-2-(oxiran-2-ylmethoxy)phenyl acetate was dissolved in MeOH (50 ml) and KOH (10% in H₂O, 40 ml) was added at 0° C. The solution was stirred for 30 min in room temperature and then evaporated to dryness. HCl (10% in H₂O) was added and the solution was extracted with EtOAc. The combined organic phases were dried (Na₂SO₄) and evaporated to dryness. The batch was mixed with another batch and then purified on flash column chromatography (isooctane/EtOAc) to give the title compound (9.3 g). MS m/z (rel. intensity, 70 eV) 196 (M+, bp), 139 (18), 125 (14), 110 (29), 69 (16).

### Preparation 21

### (7-METHOXY-2,3-DIHYDRO-1,4-BENZODIOXIN-2-YL)METHYL 4-METHYLBENZENESULFONATE

(7-methoxy-2,3-dihydro-1,4-benzodioxin-2-yl)methanol (8.8 g, 44.9 mmol) was dissolved in DCM (150 ml) and TEA (12.5 ml, 89.8 mmol) was added. The solution was cooled to 0° C. and p-toluenesulfonyl chloride (17.1 g, 89.8 mmol) dissolved in DCM (25 ml) was added dropwise. The mixture was stirred for 30 min at 0° C. and then in room temperature. Na₂CO₃ (10% in H₂O) was added and the solution was stirred. The phases were separated and organic phase dried (Na₂SO₄) and evaporated to dryness to give the title compound. Yield: 8.7 g. MS m/z (rel. intensity, 70 eV) 352 (M+, 7), 351 (M+, 20), 350 (M+, bp), 110 (10), 91 (7).

### Preparation 22

### (7-HYDROXY-2,3-DIHYDRO-1,4-BENZODIOXIN-2-YL)METHYL 4-METHYLBENZENESULFONATE

A solution of (7-methoxy-2,3-dihydro-1,4-benzodioxin-2-yl)methyl 4-methylbenzenesulfonate (8.5 g, 24.3 mmol) in DCM (200 ml) was cooled on icebath and BBr₃ (1 M in DCM, 48 ml, 48.5 mmol) was added dropwise. The solution was stirred for 45 min at 0° C. and then in room temperature. Aqueous Na₂CO₃ (10%) was added and the solution was stirred in room temperature. The phases were separated and the organic phase was dried (Na₂SO₄) and evaporated to dryness. The residue was purified on flash column chromatography (isooctane/EtOAc) to give the title compound (9.1 g). MS m/z (rel. intensity, 70 eV) 337 (M+, 19), 336 (M+, bp), 164 (28), 96 (19), 91 (27).

### Preparation 23

### {7-[(METHYLSULFONYL)OXY]-2,3-DIHYDRO-1,4-BENZODIOXIN-2-YL}METHYL 4-METHYLBENZENESULFONATE

A solution of (7-hydroxy-2,3-dihydro-1,4-benzodioxin-2-yl)methyl 4-methylbenzenesulfonate (4.5 g, 13.4 mmol) and TEA (3.7 ml, 26.8 mmol) in DCM (150 ml) was cooled to 0° C. and methanesulfonyl chloride (1.6 ml, 20.1 mmol) dissolved in DCM was added dropwise. The resulting mixture was stirred at 0° C. for 45 min and then stirred at room temperature. Na₂CO₃ (10% in H₂O) was added and the solution was stirred. The phases were separated and the organic phase was dried (Na₂SO₄) and evaporated to dryness to give the title compound (3.2 g). MS m/z (rel. intensity, 70 eV) 414 (M+, 65), 335 (bp), 163 (31), 155 (66), 91 (63).

### Preparation 24

### (7-{[(TRIFLUOROMETHYL)SULFONYL]OXY}-2,3-DIHYDRO-1,4-BENZODIOXIN-2-YL)METHYL 4-METHYLBENZENESULFONATE

Preparation according to Preparation 23: (7-hydroxy-2,3-dihydro-1,4-benzodioxin-2-yl)methyl 4-methylbenzenesulfonate (4.5 g, 13.4 mmol), DCM (150 ml), TEA (3.7 ml, 26.8 mmol), trifluorosulfonyl chloride (2.1 ml, 20.1 mmol), DCM (20 ml): Yield: 4.6 g. MS m/z (rel. intensity, 70 eV) 468 (M+, 48), 335 (bp), 163 (39), 155 (87), 91 (89).

### Preparation 25

### 3,4-DIFLUORO-2-HYDROXYBENZALDEHYDE

To a solution of magnesium methoxide (6-10% in MeOH, 40 ml, 31.1 mmol) was added 2,3-difluorophenol (6.7 g, 51.8 mmol). The mixture was heated at reflux for 40 min and MeOH (20 ml) was distilled from the solution. Toluene (50 ml+50 ml) was added and another 35 ml was distilled from the reaction mixture. Paraformaldehyde (5.6 g, 186 mmol) was added during 15 min. The resulting mixture was heated at 115° C. for 32 min and then cooled to ambient temperature. HCl (10% in H₂O) was added and the solution stirred in room temperature over night. The phases were separated, the water phase was extracted with EtOAc and the combined organic phase was washed with brine, dried (MgSO₄) and evaporated to dryness to give the title compound (5.7 g). MS m/z (rel. intensity, 70 eV) 158 (M+, 87), 157 (M+, bp), 112 (28), 101 (57), 75 (25).

### Preparation 26

### 3,4-DIFLUORO-2-(4-METHYLPHENOXY)BENZALDEHYDE

A mixture of 3,4-difluoro-2-hydroxybenzaldehyde (4.2 g, 26.4 mmol), benzylbromide (4.7 ml, 39.6 mmol) and TEA (7.3 ml, 52.7 mmol) in THF (25 ml) was put in 2 batches and heated under microwave radiation to 100° C. for 30 min. The batches were mixed and cooled to ambient temperature. HCl (10% in H₂O) and EtOAc was added. The phases were separated and the organic phase dried (MgSO₄) and purified on flash column chromatography (isooctane/EtOAc) to give the title compound (2.0 g). MS m/z (rel. intensity, 70 eV) 248 (M+, 0.1), 157 (4), 92 (8), 91 (bp), 65 (12).

### Preparation 27

### 3,4-DIFLUORO-2-(4-METHYLPHENOXY)PHENOL

A mixture of 3,4-difluoro-2-(4-methylphenoxy)benzaldehyde (2 g, 8.1 mmol) and m-CPBA (77%, 4.2 g, 24.2 mmol) in DCM (30 ml) was put in 2 batches and heated under microwave radiation to 80° C. for 1 h 10 min and then brought to ambient temperature. The batches were mixed. Aqueous Na₂CO₃ (10%) was added and the solution was extracted with EtOAc. The combined organic phases were washed with brine, dried (MgSO₄) and evaporated to dryness. Purification on flash column chromatography (isooctane/EtOAc) gave the title compound (1.3 g). MS m/z (rel. intensity, 70 eV) 236 (M+, 2), 92 (8), 91 (bp), 69 (3), 65 (11).

### Preparation 28

### 2-{[2-(BENZYLOXY)-3,4-DIFLUOROPHENOXY]METHYL}OXIRANE

A mixture of 3,4-difluoro-2-(4-methylphenoxy)phenol (1.9 g, 7.8 mmol), epibromhydrin (0.8 ml, 9.4 mmol), KOH (0.5 g, 8.6 mmol), H₂O and EtOH was heated at 80° C. for 1 h. The solution was evaporated to dryness, dissolved in EtOAc and washed with aqueous Na₂CO₃ (10%). The organic phase was dried (MgSO₄) and evaporated to dryness. Purification on flash column chromatography (isooctane/EtOAc) gave the title compound (1.6 g). MS m/z (rel. intensity, 70 eV) 292 (M+, 6), 117 (3), 92 (8), 91 (bp), 65 (9).

### Preparation 29

### (7,8-DIFLUORO-2,3-DIHYDRO-1,4-BENZODIOXIN-2-YL)METHANOL

A mixture of 2-{[2-(benzyloxy)-3,4-difluorophenoxy]methyl}oxirane (0.9 g, 3.2 mmol) and Pd/C (0.1 g) in EtOH was hydrogenated in a Parr apparatus for 1 h. The reaction mixture was filtered through a pad of celite and washed with EtOH/MeOH (1:1, 100 ml). KOH (0.7 g, 12.7 mmol) was added and the solution was stirred in room temperature over night. Aqueous HCl (10%) was added and the solution was evaporated to dryness. HCl (10% in H₂O) and EtOAc was added and the phases were separated. The organic phase was washed with brine, dried (MgSO₄) and evaporated to dryness to give the title compound (0.6 g). MS m/z (rel. intensity, 70 eV) 202 (M+, 69), 146 (bp), 145 (28), 88 (38), 57 (41).

### Preparation 30

### (7,8-DIFLUORO-2,3-DIHYDRO-1,4-BENZODIOXIN-2-YL)METHYL 4-METHYLBENZENESULFONATE

A solution of (7,8-difluoro-2,3-dihydro-1,4-benzodioxin-2-yl)methanol (0.6 g, 3.2 mmol), TEA (0.6 ml, 4.8 mmol) and p-toluenesulfonyl chloride (0.9 g, 4.8 mmol) in DCM was stirred in room temperature over night and aqueous HCl (10%) was added. The aqueous phase was extracted with EtOAc. The combined organic phases were dried (MgSO₄) and evaporated to dryness. Purification on flash column chromatography (isooctane/EtOAc) gave the title compound (0.4 g). MS m/z (rel. intensity, 70 eV) 356 (M+, 35), 184 (bp), 183 (39), 91 (71), 65 (27).

### Preparation 31

### 1-[3,5-DIFLUORO-2-(4-METHYLPHENOXY)PHENYL]ETHANONE

A solution of 1-(3,5-difluoro-2-hydroxyphenyl)ethanone (9.8 g, 57 mmol), benzylbromide (10.7 g, 7.5 ml) and K₂CO₃ (15.7 g, 114 mmol) in ACN (100 ml) was heated at reflux for 1 h and then filtered and washed with EtOAc. Purification on flash column chromatography gave the title compound (14.1 g). MS m/z (rel. intensity, 70 eV) 262 (M+, 1), 100 (6), 92 (8), 91 (bp), 65 (20).

### Preparation 32

### 3,5-DIFLUORO-2-(4-METHYLPHENOXY)PHENYL ACETATE

To a solution of 1-[3,5-difluoro-2-(4-methylphenoxy)phenyl]ethanone (6.6 g, 25.2 mmol) in CHCl₃ (dry, 30 ml) was m-CPBA (77%, 21.7 g, 125 mmol) added in portions. The resulting solution was heated at reflux over night. DCM and aqueous Na₂CO₃ (10%) was added and the phases were separated. The combined organic phases were washed with aqueous Na₂CO₃ and brine, dried (MgSO₄) and evaporated to dryness. Yield: 15.9 g. MS m/z (rel. intensity, 70 eV) 278 (M+, 1), 236 (9), 92 (8), 91 (bp), 65 (17).

### Preparation 33

### 3,5-DIFLUORO-2-(4-METHYLPHENOXY)PHENOL

A solution of 3,5-difluoro-2-(4-methylphenoxy)phenyl acetate (15.9 g), KOH (5.6 g, 99.6 mmol) in dioxan (100 ml) and H₂O (30 ml) was heated at 60° C. After 40 min further KOH (1 g) was added and the solution was heated another 30 min. The resulting solution was mixed with two other batches of the same compound. HCl (10% in H₂O), EtOAc and H₂O was added and the phases were separated. The combined organic phases were washed with brine, dried (MgSO₄) and evaporated to dryness. Purification on flash column chromatography gave the title compound. MS m/z (rel. intensity, 70 eV) 236 (M+, 1), 92 (8), 91 (bp), 89 (3), 65 (12).

### Preparation 34

### 1,5-DIFLUORO-3-METHOXY-2-(4-METHYLPHENOXY)BENZENE

A solution of 3,5-difluoro-2-(4-methylphenoxy)phenol (5.8 g, 23.3 mmol), K₂CO₃ (6.8 g, 49.1 mmol) and methyliodide (1.8 ml, 29.5 mmol) in ACN was stirred in room temperature over night, filtered and evaporated to dryness. EtOAc, H₂O and Na₂CO₃ (10% in H₂O) was added and the phases were separated. The combined organic phases were washed with brine, dried (MgSO₄) and evaporated to dryness. Yield 5.9 g. MS m/z (rel. intensity, 70 eV) 250 (M+, 6), 92 (8), 91 (bp), 88 (3), 65 (11).

### Preparation 35

### 2,4-DIFLUORO-6-METHOXYPHENOL

A mixture of 1,5-difluoro-3-methoxy-2-(4-methylphenoxy)benzene (5.9 g, 23.6 mmol), Pd/C (10%, 0.9 g), HCl (konc, 10 drops) in MeOH/EtOH was hydrogenated in a Parr apparatus for 2 h, filtered and evaporated to dryness. EtOAc and HCl (1 M) was added and the phases were separated. The combined organic phases were washed with brine, dried (MgSO₄) and evaporated to dryness. Yield: 3.3 g. MS m/z (rel. intensity, 70 eV) 160 (M+, bp), 145 (91), 117 (64), 97 (24), 69 (19).

### Preparation 36

### 2-[(2,4-DIFLUORO-6-METHOXYPHENOXY)METHYL]OXIRANE

Preparation according to Preparation 28 (heating at 70° C.): 2,4-difluoro-6-methoxyphenol (3.1 g, 19.4 mmol), epibromhydrin (1.8 ml, 21.4 mmol), KOH (1.2 g, 21.4 mmol), H₂O (10 ml), EtOH (100 ml). Yield: 3.7 g (not pure). MS m/z (rel. intensity, 70 eV) 216 (M+, 71), 160 (bp), 158 (27), 145 (70), 57 (37).

### Preparation 37

### 1-BROMO-3-(2,4-DIFLUORO-6-METHOXYPHENOXY)PROPAN-2-OL

A mixture of 2-[(2,4-difluoro-6-methoxyphenoxy)methyl]oxirane (3.6 g, 16.9 mmol) and HBr (48%, 16 ml) was heated at 100° C. over night. Further HBr (8 ml) was added and the solution was heated over night. Another 6 ml of HBr was added and after 6 h the solution was poured on ice. Et₂O was added, the phases were separated and the organic phase was evaporated to dryness with EtOH. Yield: 4.4 g. MS m/z (rel. intensity, 70 eV) 284 (M+, 7), 160 (13), 146 (bp), 145 (14), 57 (9).

### Preparation 38

### (5,7-DIFLUORO-2,3-DIHYDRO-1,4-BENZODIOXIN-2-YL)METHANOL

To a solution of 1-bromo-3-(2,4-difluoro-6-methoxyphenoxy)propan-2-ol (4.4 g) in EtOH (100 ml) and H₂O (10 ml) was added KOH (5 g). The resulting mixture was stirred in room temperature over night. HCl (10% in H₂O) was added and the solution was evaporated to dryness. Et₂O and EtOAc were added and the phases were separated. The combined organic phase was washed with brine, dried (MgSO₄) and evaporated to dryness. Yield: 3.4 g. MS m/z (rel. intensity, 70 eV) 202 (M+, bp), 171 (24), 157 (22), 146 (60), 145 (20).

### Preparation 39

### (5,7-DIFLUORO-2,3-DIHYDRO-1,4-BENZODIOXIN-2-YL)METHYL 4-METHYLBENZENESULFONATE

Preparation according to Preparation 30 (stirred in rt for 1 h): (5,7-difluoro-2,3-dihydro-1,4-benzodioxin-2-yl)methanol (3.4 g, 16.8 mmol), TEA (4 ml), p-toluenesulfonyl chloride (4.8 g, 25 2 mmol), DCM. Yield: 2.8 g. MS m/z (rel. intensity, 70 eV) 356 (M+, 73), 184 (bp), 183 (53), 155 (31), 91 (83).

### Preparation 40

### 5-FLUORO-2-[(2S)-OXIRAN-2-YLMETHOXY]BENZALDEHYDE

Preparation according to Preparation 10 (heating for 1½ h at 100° C.): 5-fluoro-2-hydroxybenzaldehyde (6.77 g, 48.3 mmol), K₂CO₃ (6.68 g, 48.4 mmol), R-glycidyltosylate (11.03 g, 48.3 mmol), DMF (50 ml). Yield: 8 g with impurities of DMF. MS m/z (rel. intensity, 70 eV) 196 (M+, 22), 139 (91), 138 (56), 83 (58), 57 (bp).

### Preparation 41

### 5-FLUORO-2-[(2S)-OXIRAN-2-YLMETHOXY]PHENYL FORMATE

Preparation according to Preparation 11 (reflux at 1 h 30 min): 5-fluoro-2-[(2S)-oxiran-2-ylmethoxy]benzaldehyde [8 g (including impurities of DMF), 40.8 mmol], m-CPBA (77%, 12.8 g, 57.1 mmol), DCM (40 ml). MS m/z (rel. intensity, 70 eV) 212 (M+, 10), 184 (78), 139 (bp), 128 (75), 57 (56).

### Preparation 42

### [(2S)-7-FLUORO-2,3-DIHYDRO-1,4-BENZODIOXIN-2-YL]METHANOL

A mixture of 5-fluoro-2-[(2S)-oxiran-2-ylmethoxy]phenyl formate (2.2 g, 10.4 mmol) and aqueous sodium hydroxide (15%, 10 ml) was heated at reflux for 1 h 30 min. The mixture was cooled to ambient temperature and extracted with Et₂O. The organic phase was washed with H₂O, dried (MgSO₄), evaporated to dryness and purified on flash column chromatography (EtOAc/MeOH) to give the title compound. MS m/z (rel. intensity, 70 eV) 184 (M+, bp), 153 (35), 138 (23), 128 (73), 127 (25).

### Preparation 43

### [(2R)-7-FLUORO-2,3-DIHYDRO-1,4-BENZODIOXIN-2-YL]METHYL 4-METHYLBENZENESULFONATE

To a solution of [(2S)-7-fluoro-2,3-dihydro-1,4-benzodioxin-2-yl]methanol (0.12 g, 0.64 mmol) in DCM (10 ml) was added dibutyltinoxide (3 mg, 0.012 mmol), p-toluenesulfonyl chloride (0.12 g, 0.64 mmol) and TEA (0.089 ml, 0.64 mmol). The mixture was stirred at room temperature for 15 h and then mixed with another batch of the same compound. Water and EtOAc were added and the phases were separated. The combined organic phases were dried (MgSO₄) and evaporated to dryness to give the title compound. Yield: 0.50 g. MS m/z (rel. intensity, 70 eV) 338 (M+, 82), 166 (bp), 165 (56), 139 (33), 91 (58).

### Preparation 44

### 2-[4-(TRIFLUOROMETHYL)PHENOXY]TETRAHYDRO-2H-PYRAN

To a mixture of 4-(trifluoromethyl)phenol (0.5 g, 3.08 mmol), HCl in 1,4-dioxane (10 ml, 4 N), and DCM (30 ml) was added 3,4-dihydro-2H-pyran (0.65 g, 7.7 mmol). The mixture was stirred overnight at room temperature. Aqueous NaHCO₃ (sat.) was added and the organic phase was separated, dried (MgSO₄) and evaporated to dryness. Flash column chromatography (isooctane/EtOAc) yielded the title compound. Yield: 0.65 g. MS m/z (rel. intensity, 70 eV) 162 (14), 143 (17), 85 (bp), 67 (24), 57 (23).

### Preparation 45

### 2-HYDROXY-5-(TRIFLUOROMETHYL)BENZALDEHYDE

n-BuLi (1.7 ml, 42 mmol) was added to a mixture of TMEDA (0.6 ml) and THF (25 ml) at −10° C. under N₂. After 30 min a solution of 2-[4-(trifluoromethyl)phenoxy]tetrahydro-2H-pyran (0.7 g, 2.84 mmol) in dry THF (5 ml) was added dropwise. The mixture was stirred for 15 min at −10° C. and was then brought to room temperature. HCl (22% in water) was added, the organic phase was separated and was added to HCl in 1,4-dioxane (15 ml, 4N) and the resulting mixture was stirred at room temperature overnight. Water (50 ml) was added, the organic phase was separated, dried (MgSO₄) and evaporated to dryness. Flash column chromatography (isooctane/EtOAc) yielded the title compound. Yield: 0.12 g. MS m/z (rel. intensity, 70 eV) 190 (M+, 90), 189 (bp), 161 (33), 144 (28), 63 (23).

### Preparation 46

### 2-[(2R)-OXIRAN-2-YLMETHOXY]-5-(TRIFLUOROMETHYL)BENZALDEHYDE

A mixture of 2-hydroxy-5-(trifluoromethyl)benzaldehyde (1.0 g, 5.25 mmol), (R)-glycidyltosylate (1.05 g, 4.6 mmol), K₂CO₃ (1.3 g, 9.4 mmol) and DMF was stirred overnight at 60° C. The mixture was evaporated to dryness and EtOAc was added. The organic phase was washed with HCl (20 ml, 1 N), dried (MgSO₄) and evaporated to dryness. Flash column chromatography (isooctane/EtOAc) yielded the title compound. Yield 0.58 g. MS m/z (rel. intensity, 70 eV) 246 (M+, 3), 228 (33), 189 (bp), 188 (82), 160 (37).

### Preparation 47

### [(2S)-7-(TRIFLUOROMETHYL)-2,3-DIHYDRO-1,4-BENZODIOXIN-2-YL]METHANOL

A mixture of 2-[(2R)-oxiran-2-ylmethoxy]-5-(trifluoromethyl)benzaldehyde (0.58 g, 2.36 mmol) m-CPBA (77%, 0.61 g, 3.5 mmol) and CHCl₃ (10 ml) was stirred overnight at reflux. m-CPBA (77%, 0.30 g, 1.7 mmol) was added and the mixture was refluxed for 3 h. Aqueous NaHCO₃ (sat.) was added and the organic phase was separated, washed with brine, dried (MgSO₄) and evaporated to dryness. KOH (10 ml, 10% in H₂O) and 1,4-dioxane (10 ml) was added and the mixture was stirred for 1 h at room temperature. Aqueous NaHCO₃ (sat.) was added and the organic phase was separated, washed with brine, dried (MgSO₄) and evaporated to yield the title compound (0.40 g). MS m/z (rel. intensity, 70 eV) 234 (M+, 79, 203 (bp), 189 (37), 178 (93), 57 (67).

### Preparation 48

### [(2R)-7-(TRIFLUOROMETHYL)-2,3-DIHYDRO-1,4-BENZODIOXIN-2-YL]METHYL 4-METHYLBENZENESULFONATE

A mixture of [(2S)-7-(trifluoromethyl)-2,3-dihydro-1,4-benzodioxin-2-yl]methanol (0.40 g, 1.71 mmol), p-toluenesulfonyl chloride (0.35 g, 1.71 mmol), TEA (0.23 ml, 1.71 mmol) and 4-DMAP (0.21 g, 1.71 mmol) in DCM (15 ml) was stirred at room temperature for 1 h and 30 min. HCl (1 N) was added, the organic phase was separated, washed with brine, dried (MgSO₄) and concentrated to give the title compound (0.48 g). MS m/z (rel. intensity, 70 eV) 388 (M+, 27), 216 (bp), 215 (46), 203 (23), 91 (47).

### Preparation 49

### 1-[2-(BENZYLOXY)-4-FLUOROPHENYL]ETHANONE

A mixture of 1-(4-fluoro-2-hydroxyphenyl)ethanone (2.7 g, 17.5 mmol), benzylbromide (3.60 g, 21.0 mmol) and K₂CO₃ (3.63 g, 26.3 mmol) was stirred in dry DMF (15 ml) under N₂ at 80° C. overnight. The solution was brought to ambient temperature and water and EtOAc was added. The water phase was extracted with EtOAc. The combined organic phases were washed with LiCl (5%) and evaporated to dryness to give the crude title compound (4.5 g). MS m/z (rel. intensity, 70 eV) 244 (M+, 18), 139 (9), 92 (17), 91 (bp), 65 (26).

### Preparation 50

### ETHYL 4-[2-(BENZYLOXY)-4-FLUOROPHENYL]-2,4-DIOXOBUTANOATE

Sodium (2.15 g, 93.3 mmol) was dissolved in EtOH and the solution was filtrated and added to a mixture of 1-[2-(benzyloxy)-4-fluorophenyl]ethanone (4.56 g, 18.7 mmol) and diethyloxalate (12.63 ml, 93.3 mmol) in EtOH (200 ml). The mixture was heated at 80° C. for 2.5 h, followed by evaporation of EtOH, addition of HCl (10% in H₂O) and addition of EtOAc. The phases were separated, the organic phase was dried (Na₂SO₄), filtered and concentrated. The residue was purified by flash column chromatography (isooctane/EtOAc) to give the title compound (5.3 g). MS m/z (rel. intensity, 70 eV) 344 (M+, 1), 271 (16), 139 (11), 92 (11), 91 (bp), 65 (11).

### Preparation 51

### ETHYL 4-(4-FLUORO-2-HYDROXYPHENYL)-2-HYDROXYBUTANOATE

A mixture of ethyl 4-[2-(benzyloxy)-4-fluorophenyl]-2,4-dioxobutanoate (5.08 g, 14.75 g), palladium black (2.5 g) and EtOH was hydrogenated at 40 Psi for 3 h. The mixture was filtrated through Celite, palladium black (2.5 g) was added and the mixture was again hydrogenated at 40 Psi for 3+3 h. The procedure was repeated using palladium black (1.9 g) and hydrogenation at 40 Psi for 40 h. Filtration through Celite and evaporation gave the title compound (2.8 g). MS m/z (rel. intensity, 70 eV) 242 (M+, 24), 149 (27), 125 (bp), 104 (33), 76 (21).

### Preparation 52

### ETHYL 7-FLUOROCHROMANE-2-CARBOXYLATE

Ethyl 4-(4-fluoro-2-hydroxyphenyl)-2-hydroxybutanoate (2.20 g, 9.1 mmol) and triphenylphosphine (2.62 g, 10.0 mmol) were dissolved in dry THF (5 ml), using a sonic bath. Diispropylhydrazine-1,2-dicarboxylate (2.02 g, 10.0 mmol) was added and the mixture was stirred for 4 h. Water was added and the water phase was extracted with EtOAc. The combined organic phases were evaporated to dryness. Purification by flash chromatography (isooctane/EtOAc) gave the title compound (1.3 g). MS m/z (rel. intensity, 70 eV) 224 (M+, 74), 178 (32), 151 (bp), 149 (38), 123 (30).

### Preparation 53

### (7-FLUORO-3,4-DIHYDRO-2H-CHROMEN-2-YL)METHANOL

LiAlH₄ (0.41 g, 10.7 mmol) was added to ethyl 7-fluorochromane-2-carboxylate (1.2 g, 5.4 mmol) in THF (8 ml) at 0° C. The mixture was stirred at room temperature for 1 h. EtOH was added, followed by HCl (10% in H₂O) and EtOAc. The organic phase was separated, washed with brine, dried (Na₂SO₄), filtered and concentrated to give the title compound. Yield 0.90 g. MS m/z (rel. intensity, 70 eV) 182 (M+, 57), 151 (bp), 125 (40), 123 (32), 103 (32).

### Preparation 54

### (7-FLUORO-3,4-DIHYDRO-2H-CHROMEN-2-YL)METHYL 4-METHYLBENZENESULFONATE

A mixture of (7-fluoro-3,4-dihydro-2H-chromen-2-yl)methanol (0.9 g, 4.9 mmol), p-toluenesulfonyl chloride (1.41 g, 7.4 mmol), TEA (0.83 ml, 5.9 mmol), 4-DMAP (0.72 g, 5.9 mmol) and DCM (25 ml) was stirred at room temperature for 2 h. Water and DCM was added and the phases were separated. The organic phase was washed with brine, dried (Na₂SO₄), filtered and concentrated. Purification by flash chromatography (isooctane/EtOAc) gave the title compound (1.15 g). MS m/z (rel. intensity, 70 eV) 336 (M+, 57), 164 (bp), 163 (93), 151 (72), 149 (82), 91 (73).

### Biological Activity

The following tests are used for evaluation of the compounds according to the invention.

### In Vivo Test: Behaviour

Behavioural activity is measured using eight Digiscan activity monitors (RXYZM (16) TAO, Omnitech Electronics, Columbus, Ohio, USA), connected to an Omnitech Digiscan analyzer and an Apple Macintosh computer equipped with a digital interface board (NB DIO-24, National Instruments, USA). Each activity monitor consists of a quadratic metal frame (W×L=40 cm×40 cm) equipped with photobeam sensors. During measurements of behavioural activity, a rat is put in a transparent acrylic cage (W×L×H, 40×40×30 cm) which in turn is placed in the activity monitor. Each activity monitor is equipped with three rows of infrared photobeam sensors, each row consisting of 16 sensors. Two rows are placed along the front and the side of the floor of the cage, at a 90° angle, and the third row is placed 10 cm above the floor to measure vertical activity. Photobeam sensors are spaced 2.5 cm apart. Each activity monitor is fitted in an identical sound and light attenuating box containing a weak house light and a fan.

The computer software is written using object oriented programming (LabVIEW®, National instruments, Austin, Tex., USA).

Behavioural data from each activity monitor, representing the position (horizontal center of gravity and vertical activity) of the animal at each time, are recorded at a sampling frequency of 25 Hz and collected using a custom written LABView™ application. The data from each recording session are stored and analyzed with respect to distance traveled. Each behavioural recording session lasts 60 min, starting approximately 4 min after the injection of test compound. Similar behavioural recording procedures are applied for drug-naïve and drug pre-treated rats. Rats pre-treated with d-amphetamine are given a dose of 1.5 mg/kg i.p. 10 min before the recording session in the activity monitor. Rats pre-treated with MK-801 are given a dose of 0.7 mg/kg i.p. 90 min before the recording session in the activity monitor. The results are presented as counts/60 minutes, or counts/30 minutes, in arbitrary length units. Statistical comparisons are carried out using Student's t-test against the control group. In MK-801 or amphetamine pre-treated animals, statistical comparisons are made against the MK801 or d-amphetamine controls, respectively.

ED₅₀ values for reduction of amphetamine-induced hyper-locomotion are calculated by curve fitting. For most compounds, the evaluation is based on 16 amphetamine pre-treated animals over the dose range 0, 11, 33 and 100 μmol/kg s.c. in one single experiment, with complementary doses in separate experiments. Calculations are based on distance during the last 45 minutes of one hour of measurement. The distances are normalised to amphetamine-control and fitted by least square minimization to the function “End-(End-Control)/(1+(dose/ED₅₀)^{Slope})”. The four parameters (Control, End, ED₅₀ and Slope) are fitted with the restrictions: ED₅₀>0, 0.5<Slope<3, End=0% of control. The restriction with locked End is made to focus on potency rather than efficacy. To estimate confidence levels for the parameters, the fit is repeated 100 times with a random evenly distributed squared weight (0 to 1) for every measurement value. Presented ED₅₀-ranges cover 95% of these values.

### In Vivo Test: Neurochemistry

After the behavioural activity sessions, the rats are decapitated and their brains rapidly taken out and put on an ice-cold petri-dish. The limbic forebrain, the striatum, the frontal cortex and the remaining hemispheral parts of each rat are dissected and frozen. Each brain part is subsequently analyzed with respect to its content of monoamines and their metabolites.

The monoamine transmitter substances (NA (noradrenaline), DA (dopamine), 5-HT (serotonin)) as well as their amine (NM (normethanephrine), 3-MT (3-methoxytyramine)) and acid (DOPAC (3,4-dihydroxyphenylacetic acid), 5-HIAA (5-hydroxyindoleacetic acid), HVA (homovanillic acid)) metabolites are quantified in brain tissue homogenates by HPLC separations and electrochemical detection

The analytical method is based on two chromatographic separations dedicated for amines or acids. Two chromatographic systems share a common auto injector with a 10-port valve and two sample loops for simultaneous injection on the two systems. Both systems are equipped with a reverse phase column (Luna C18(2), dp 3 μm, 50*2 mm i.d., Phenomenex) and electrochemical detection is accomplished at two potentials on glassy carbon electrodes (MF-1000, Bioanalytical Systems, Inc.). The column effluent is passed via a T-connection to the detection cell or to a waste outlet. This is accomplished by two solenoid valves, which block either the waste or detector outlet. By preventing the chromatographic front from reaching the detector, better detection conditions are achieved. The aqueous mobile phase (0.4 ml/min) for the acid system contains citric acid 14 mM, sodium citrate 10 mM, MeOH 15% (v/v) and EDTA 0.1 mM. Detection potentials relative to Ag/AgCl reference are 0.45 and 0.60V. The aqueous ion pairing mobile phase (0.5 ml/min) for the amine system contains citric acid 5 mM, sodium citrate 10 mM, MeOH 9% (v/v), MeCN 10.5% v/v), decane sulfonic acid 0.45 mM, and EDTA 0.1 mM. Detection potentials relative to Ag/AgCl reference are 0.45 and 0.65V.

ED₅₀ values for the increase of DOPAC in striatum are calculated by curve fitting. For most compounds, the evaluation is based on 20 animals over the dose range 0, 3.7, 11, 33 and 100 μmol/kg s.c. in one single experiment, with complementary doses in separate experiments. The DOPAC levels are normalised to control and fitted by least square minimization to the function “End-(End-Control)/(1+(dose/ED₅₀)^{Slope})”. The four parameters (Control, End, ED₅₀ and Slope) are fitted with the restrictions: ED₅₀>0, 0.5<Slope<3, 350<End<400% of control. To estimate confidence levels for the parameters, the fit is repeated 100 times with a random evenly distributed squared weight (0 to 1) for every measurement value. Presented ED₅₀-ranges cover 95% of these values.

### In Vivo Test: Oral Bioavailability

Experiments are performed 24 hours after implantation of arterial and venous catheters. Test compound is administered orally at 12.5 μmol/kg or intravenously at 5 μmol/kg using the venous catheters, n=3 per group. Arterial blood samples are then taken during six hours at 0, 3, 9, 27, 60, 120, 180, 240, 300 and, 360 minutes after administration of the test compound. The oral bioavailability is calculated as the ratio of the AUC (Area under curve) obtained after oral administration over the AUC obtained after intravenous administration for each rat. The parameter AUC is calculated according to the following:

AUC: the area under the plasma concentration versus time curve from time zero to the last concentration measured (Clast), calculated by the log/linear trapezoidal method.

The levels of test compound are measured by means of liquid chromatography-mass spectrometry (LC-MS) (Hewlett-Packard 1100MSD Series). The LC-MS module includes a quaternary pump system, vacuum degasser, thermostatted autosampler, thermostatted column compartment, diode array detector and API-ES spray chamber. Data handling was performed with a HP ChemStation rev.A.06.03. system. Instrument settings: MSD mode: Selected ion monitoring (SIM) MSD polarity: Positiv Gas temp: 350° C. Drying gas: 13.0 l/min Nebulizer gas: 50 psig Capillary voltage: 5000 V Fragmentor voltage: 70 V.

Analytical column: Zorbax eclipse XDB-C8 (4.6*150 mm, 5 μm) at 20° C. The mobile phase is acetic acid (0.03%) (solvent A) and acetonitrile (solvent B). The flow rate of the mobile phase is 0.8 ml/min. The elution is starting at 12% of solvent β isocratic for 4.5 min, then increasing linearity to 60% over 4.5 min.

Extractions procedure: Plasma samples (0.25-0.5 ml) are diluted with water to 1 ml, and 60 pmol (100 μl) internal standard (−)-OSU6241 is added. The pH was adjusted to 11 by the addition of 25 μl saturated Na₂CO₃. After mixing, the samples are extracted with 4 ml dichloromethane by shaking for 20 min. The organic layer is after centrifugation transferred to a smaller tube and evaporated to dryness under a stream of nitrogen. The residue is then dissolved in 120 μl mobile phase (acetic acid (0.03%): acetonitrile, 95:5) for LC-MS analysis (10 μl injected). The selective ion (MH⁺) is monitored for each Example, and MH⁺296 for (−)-OSU6241 ((3-[3-(ethylsulfonyl)phenyl]-1-propylpiperidine).

A standard curve over the range of 1-500 pmol is prepared by adding appropriate amounts of test compound to blank plasma samples.

### In Vitro Test: Metabolic Stability in Rat Liver Microsomes

Rat liver microsomes are isolated as described by Förlin [Förlin L: Effects of Clophen A50, 3-methylcholantrene, pregnenolone-16aq-carbonitrile and Phenobarbital on the hepatic microsomal cytochrome P-450-dependent monooxygenaser system in rainbow trout, salmo gairdneri, of different age and sex; Tox Appl Pharm. 1980 54 (3) 420-430] with minor modifications e.g. 3 mL/g liver of a 0.1 M Na/K*PO₄ buffer with 0.15M KCl, pH 7.4, (buffer 1) is added before homogenisation, the homogenate is centrifuged for 20 minutes instead of 15, the supernatant is ultracentrifuged at 100.000 g instead of 105.000 g and the pellet from the ultracentrifugation is resuspended in 1 mL/g liver of 20% v/v 87% glycerol in buffer 1.

1 μL of, 0.2 or 1 mM test substance diluted in water, and 10 μL 20 mg/mL rat liver microsome are mixed with 149 μL 37° C. buffer 1 and the reaction is started by addition of 40 μL 4.1 mg/mL NADPH. After 0 or 15 minutes incubation at 37° C. in a heating block (LAB-LINE, MULTI-BLOK Heater or lab4you, TS-100 Thermo shaker at 700 rpm) the reaction is stopped by addition of 100 μL pure acetonitrile. The protein precipitation is then removed by rejecting the pellet after centrifugation at 10.000 g for 10 minutes (Heraeus, Biofuge fresco) in 4° C. The test compound is analysed using HPLC-MS (Hewlett-Packard 1100MSD Series) with a Zorbax SB-C18 column (2.1*150 mm, 5 μm) using 0.03% formic acid and acetonitrile as mobile phase (gradient) or a Zorbax Eclipse XDB-C18 (3*75 mm, 3.5 μm) using 0.03% acetic acid and acetonitrile as mobile phase (gradient). The 15 min turnover is calculated as the fraction of test compound eliminated after 15 minutes, expressed in percent of 0 min levels, i.e. 100*[conc test compound at 0 min−concentration at 15 min]/conc at 0 min.

Preparation of liver microsomes is performed as described in Förlin [Förlin L: Effects of Clophen A50, 3-methylcholantrene, pregnenolone-16aq-carbonitrile and Phenobarbital on the hepatic microsomal cytochrome P-450-dependent monooxygenaser system in rainbow trout, salmo gairdneri, of different age and sex; Tox Appl Pharm. 1980 54 (3) 420-430]. Protocols for incubation with liver microsomes are referred in Crespi et Stresser [Crespi C L, DM Stressser: Fluorometric screening for metabolism based drug-drug interactions; J. Pharm. Tox. Meth. 2000 44 325-331], and Renwick et al. [Renwick A B et al.: Metabolism of 2,5-bis(trifluoromethyl)-7-benzyloxy-4-trifluoromethylcoumarin by human hepatic CYP isoforms: evidence for selectivity towards CYP3A4; Xenobiotica 2001 31 (4) 187-204].

### Microdialysis

Male Sprague-Dawley rats weighing 220-320 g are used throughout the experiments. Before the experiment the animals are group housed, five animals in each cage, with free access to water and food. The animals are housed at least one week after arrival prior to surgery and use in the experiments. Each rat is used only once for microdialysis.

We use a modified version Waters et al. [Waters N, Lofberg L, Haadsma-Svensson S, Svensson K, Sonesson C and Carlsson A: Differential effects of dopamine D2 and D3 receptor antagonists in regard to dopamine release, in vivo receptor displacement and behaviour; J. Neural. Transm. Gen. Sect. 1994 98 (1) 39-55] of the I-shaped probe as described by Santiago and Westerink [Santiago M, Westerink B H C: Characterization of the in vivo release of dopamine as recorded by different types of intracerebral microdialysis probes; Naunyn-Schmiedeberg's Arch. Pharmacol. 1990 342 407-414]. The dialysis membrane we use is the AN69 polyacrylonitrile/sodiummethalylsulfonate copolymer (HOSPAL; o.d./i.d. 310/220 μm: Gambro, Lund, Sweden). In the dorsal striatum we use probes with an exposed length of 3 mm of dialysis membrane and in the prefrontal cortex the corresponding length is 2.5 mm. The rats are operated under isoflurane inhalationanesthesia while mounted into a Kopf stereotaxic instrument. Co-ordinates are calculated relative to bregma; dorsal striatum AP+1, ML±2.6, DV−6.3; Pf cortex, AP+3.2, 8° ML±1.2, DV−4.0 according to Paxinos and Watson [Paxinos G, Watson C: The Rat Brain in Stereotaxic Coordinates; New York, Academic Press 1986]. The dialysis probe is positioned in a burr hole under stereotaxic guidance and cemented with phosphatine dental cement. The rats are housed individually in cages for 48 h before the dialysis experiments, allowing them to recover from surgery and minimizing the risk of drug interactions with the anaesthetic during the following experiments. During this period the rats have free access to food and water. On the day of experiment the rats are connected to a micro perfusion pump via a swivel and are replaced in the cage where they can move freely within its confinements. The perfusion medium is a Ringer's solution containing in mmol/l: NaCl; 140, CaCl2; 1.2, KCl; 3.0, MgCl2; 1.0 and ascorbic acid; 0.04 according to Moghaddam and Bunney [Moghaddam B, Bunney B S: Ionic Composition of Microdialysis Perfusing Solution Alters the Pharmacological Responsiveness and Basal Outflow of Striatal Dopamine; J. Neurochem. 1989 53 652-654]. The pump is set to a perfusion speed of 2 μl/min and 40 μl samples are collected every 20 min.

Each sample is analyzed at two HPLC systems. On an autoinjector (CMA 200) with a 10-port valve (Valco C10WE), holding two sample loops in series (4 μl and 20 μl), each brain dialysate sample is loaded in both loops simultaneously. At injection the 20 μl sample is introduced into a column switching system (reverse-phase combined with reverse-phase ion-pairing) for dopamine (DA), noradrenaline (NA), normetanephrine (NM), 3-methoxytyramine (3-MT) and serotonin (5-hydroxytryptamine, 5-HT) determination, while the 4 μl sample is introduced on a reverse-phase column for the chromatography of the acidic monoamine metabolites 3,4-di-hydroxyphenylacetic acid (DOPAC), homovanillic acid (HVA) and 5-hydroxyindoleacetic acid (5-HIAA). The currents generated by the two EC detectors are converted to digital data and evaluated using Chromeleon software (Dionex) on a PC. The method sample turn over time is 4.5 min and two parallel experiments are normally analyzed simultaneously on the system. After the experiment the rats are uncoupled from the perfusion pump and decapitated. Their brains are rapidly taken out and fixed in Neo-fix solution (Kebo-lab, Sweden) for subsequent inspection of probe localisation. The Animal Ethics Committee in Göteborg, Sweden approved the procedures applied in these experiments.

1. A compound of Formula 1: 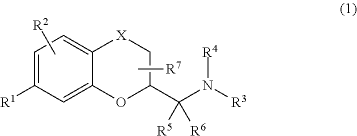
any of its stereoisomers or any mixture of its stereoisomers, or an N-oxide thereof, or a pharmaceutically acceptable salt thereof, wherein
X is O, S, NH or CH₂;
R¹ is selected from the group consisting of OSO₂CF₃, OSO₂CH₃, COR⁸, CN, OCF₃, SCF₃, OCHF₂, SCHF₂, CF₃, F, Cl, Br, I, SF₅, SCN, OCN, OCOCF₃, SCOCF₃, OCOCH₃, SCOCH₃ and CH(OH)CF₃;
R² is selected from the group consisting of H, CN, F, Cl, Br, I and CH₃;
R³ is selected from the group consisting of C₁-C₅ alkyl, allyl, CH₂CH₂OCH₃, CH₂CH₂CH₂F, CH₂CH₂CHF₂, CH₂CH₂F, 3,3,3-trifluoropropyl, 4,4,4-trifluorobutyl, CH₂CH₂OH, CH₂CH₂CH₂OH, CH₂CH(OH)CH₃, CH₂CH₂COCH₃, C₃-C₆ cycloalkyl, 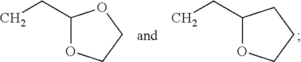
R⁴ is selected from the group consisting of H and C₁-C₅ alkyl; or
R³ and R⁴ together with the nitrogen atom to which they are attached form a four- to six-membered heterocyclic ring, which heterocyclic ring may optionally comprise as a ring member, one oxygen atom, and/or one additional nitrogen atom; and which heterocyclic ring may optionally be substituted with C₁-C₅ alkyl;
R⁵, R⁶ and R⁷ are selected from the group consisting of H and CH₃; and
R⁸ is selected from the group consisting of C₁-C₃ alkyl, CF₃, CHF₂, CH₂F and CN.

2. The compound according to claim 1, any of its stereoisomers or any mixture of its stereoisomers, or an N-oxide thereof, or a pharmaceutically acceptable salt thereof; wherein X is O, S, NH or CH₂.

3. The compound according to claim 1, any of its stereoisomers or any mixture of its stereoisomers, or an N-oxide thereof, or a pharmaceutically acceptable salt thereof, wherein R¹ is selected from the group consisting of OSO₂CF₃, OSO₂CH₃, COR⁸, CN, OCF₃, SCF₃, OCHF₂, SCHF₂, CF₃, F, Cl, Br, I, SF₅, SCN, OCN, OCOCF₃, SCOCF₃, OCOCH₃, SCOCH₃ and CH(OH)CF₃; and
R⁸ is selected from the group consisting of C₁-C₃ alkyl, CF₃, CHF₂, CH₂F and CN.

4. A compound according to claim 1, any of its stereoisomers or any mixture of its stereoisomers, or an N-oxide thereof; or a pharmaceutically acceptable salt thereof, wherein R² is selected from the group consisting of H, CN, F, Cl, Br, I and CH₃.

5. A compound according to claim 1, any of its stereoisomers or any mixture of its stereoisomers, or an N-oxide thereof, or a pharmaceutically acceptable salt thereof; wherein R³ is selected from the group consisting of C₁-C₅ alkyl, allyl, CH₂CH₂OCH₃, CH₂CH₂CH₂F, CH₂CH₂CHF₂, CH₂CH₂F, 3,3,3-trifluoropropyl, 4,4,4-trifluorobutyl, CH₂CH₂OH, CH₂CH₂CH₂OH, CH₂CH(OH)CH₃, CH₂CH₂COCH₃, C₃-C₆ cycloalkyl,

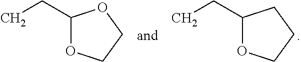

6. The compound according to claim 1, any of its stereoisomers or any mixture of its stereoisomers, or an N-oxide thereof; or a pharmaceutically acceptable salt thereof; wherein R⁴ is selected from the group consisting of H and C₁-C₅ alkyl.

7. The compound according to claim 1, any of its stereoisomers or any mixture of its stereoisomers, or an N-oxide thereof, or a pharmaceutically acceptable salt thereof, wherein R³ and R⁴ together with the nitrogen atom to which they are attached form a four- to six-membered heterocyclic ring, which heterocyclic ring may optionally comprise as a ring member, one oxygen atom, and/or one additional nitrogen atom; and which heterocyclic ring may optionally be substituted with C₁-C₅ alkyl.

8. The compound according to claim 1, any of its stereoisomers or any mixture of its stereoisomers, or an N-oxide thereof, or a pharmaceutically acceptable salt thereof, wherein R⁵, R⁶ and R⁷ are selected from the group consisting of H and CH₃.

9. The compound according to claim 1, any of its stereoisomers or any mixture of its stereoisomers, or an N-oxide thereof, or a pharmaceutically acceptable salt thereof, wherein
X is O;
R¹ is OSO₂CF₃, OSO₂CH₃, CF₃, F, Cl, Br;
R² is H, F;
R³ is C₁-C₅ alkyl, allyl or CH₂CH₂OH; and
R⁴ is H and C₁-C₅ alkyl; or
R³ and R⁴ together the nitrogen atom to which they are attached form an acetidine, a pyrrolidine, a piperidine or a morpholine group; and
R⁵, R⁶ and R⁷ are selected from the group consisting of H and CH₃.

10. The compound according to claim 1, which is
N-{[(2S)-7-BROMO-2,3-DIHYDRO-1,4-BENZODIOXIN-2-YL]METHYL}PROPAN-1-AMINE;
N-[(6,7-DIFLUORO-2,3-DIHYDRO-1,4-BENZODIOXIN-2-YL)METHYL]PROPAN-1-AMINE;
N-[(7-FLUORO-2,3-DIHYDRO-1,4-BENZODIOXIN-2-YL)METHYL]PROPAN-1-AMINE;
N-[(7-CHLORO-2,3-DIHYDRO-1,4-BENZODIOXIN-2-YL)METHYL]PROPAN-1-AMINE;
3-[(PROPYLAMINO)METHYL]-2,3-DIHYDRO-1,4-BENZODIOXIN-6-YL METHANESULFONATE;
3-[(PROPYLAMINO)METHYL]-2,3-DIHYDRO-1,4-BENZODIOXIN-6-YL TRIFLUOROMETHANESULFONATE;
N-[7,8-DIFLUORO-2,3-DIHYDRO-1,4-BENZODIOXIN-2-YL)METHYL]PROPAN-1-AMINE;
N-[5,7-DIFLUORO-2,3-DIHYDRO-1,4-BENZODIOXIN-2-YL)METHYL]PROPAN-1-AMINE;
1-{[(2S)-7-FLUORO-2,3-DIHYDRO-1,4-BENZODIOXIN-2-YL]METHYL}PYRROLIDINE;
1-[(7-FLUORO-2,3-DIHYDRO-1,4-BENZODIOXIN-2-YL)-N-METHYLMETHANAMINE;
N-[(7-FLUORO-2,3-DIHYDRO-1,4-BENZODIOXIN-2-YL)METHYL]BUTAN-1-AMINE;
2-{[(7-FLUORO-2,3-DIHYDRO-1,4-BENZODIOXIN-2-YL)METHYL]AMINO}ETHANOL;
N-[(7-FLUORO-2,3-DIHYDRO-1,4-BENZODIOXIN-2-YL)METHYL]-N-PROPYLPROPAN-1-AMINE;
N-ETHYL-N-[(7-FLUORO-2,3-DIHYDRO-1,4-BENZODIOXIN-2-YL)METHYL]PROPAN-1-AMINE;
1-[(7-FLUORO-2,3-DIHYDRO-1,4-BENZODIOXIN-2-YL)METHYL]PIPERIDINE;
N-[(7-FLUORO-2,3-DIHYDRO-1,4-BENZODIOXIN-2-YL)METHYL]ETHANAMINE;
N-[7-FLUORO-2,3-DIHYDRO-1,4-BENZODIOXIN-2-YL)METHYL]PROP-2-EN-1-AMINE;
1-[(7-FLUORO-2,3-DIHYDRO-1,4-BENZODIOXIN-2-YL)-N,N-DIMETHYLMETHANAMINE;
N-[(7-FLUORO-2,3-DIHYDRO-1,4-BENZODIOXIN-2-YL)METHYL]-N-METHYLPROPAN-1-AMINE;
1-[(7-FLUORO-2,3-DIHYDRO-1,4-BENZODIOXIN-2-YL)METHYL]AZETIDINE;
4-[(7-FLUORO-2,3-DIHYDRO-1,4-BENZODIOXIN-2-YL)METHYL]MORPHOLINE;
N-[(7-FLUORO-2,3-DIHYDRO-1,4-BENZODIOXIN-2-YL)METHYL]-2-METHOXYETHANAMINE;
N-ETHYL-N-[(7-FLUORO-2,3-DIHYDRO-1,4-BENZODIOXIN-2-YL)METHYL]ETHANAMINE;
N-[(7-FLUORO-2,3-DIHYDRO-1,4-BENZODIOXIN-2-YL)METHYL]-N-METHYLETHANAMINE;
3-[(METHYLAMINO)METHYL]-2,3-DIHYDRO-1,4-BENZODIOXIN-6-YL METHANESULFONATE;
3-[(ETHYLAMINO)METHYL]-DIHYDRO-1,4-BENZODIOXIN-6-YL METHANESULFONATE;
3-[(BUTYLAMINO)METHYL]-2,3-DIHYDRO-1,4-BENZODIOXIN-6-YL METHANESULFONATE;
3-{[(2-HYDROXYETHYL)AMINO]METHYL}-2,3-DIHYDRO-1,4-BENZODIOXIN-6-YL METHANESULFONATE;
3-[(DIPROPYLAMINO)METHYL]-2,3-DIHYDRO-1,4-BENZODIOXIN-6-YL METHANESULFONATE;
3-{[ETHYL(PROPYL)AMINO]METHYL}-2,3-DIHYDRO-1,4-BENZODIOXIN-6-YL METHANESULFONATE;
3-(PIPERIDIN-1-YLMETHYL)-2,3-DIHYDRO-1,4-BENZODIOXIN-6-YL METHANESULFONATE;
3-[(DIMETHYLAMINO)METHYL]-2,3-DIHYDRO-1,4-BENZODIOXIN-6-YL METHANESULFONATE;
3-{[METHYL(PROPYL)AMINO]METHYL}-2,3-DIHYDRO-1,4-BENZODIOXIN-6-YL METHANESULFONATE;
3-(MORPHOLIN-4-YLMETHYL)-2,3-DIHYDRO-1,4-BENZODIOXIN-6-YL METHANESULFONATE;
3-[(DIETHYLAMINO)METHYL]-2,3-DIHYDRO-1,4-BENZODIOXIN-6-YL METHANESULFONATE;
3-(PYRROLIDIN-1-YLMETHYL)-2,3-DIHYDRO-1,4-BENZODIOXIN-6-YL METHANESULFONATE;
3-[(ALLYLAMINO)METHYL]-2,3-DIHYDRO-1,4-BENZODIOXIN-6-YL METHANESULFONATE;
3-{[ETHYL(METHYL)AMINO]METHYL}-2,3-DIHYDRO-1,4-BENZODIOXIN-6-YL METHANESULFONATE;
3-{[(2-METHOXYETHYL)AMINO]METHYL}-2,3-DIHYDRO-1,4-BENZODIOXIN-6-YL METHANESULFONATE;
3-(AZETIDIN-1-YLMETHYL)-2,3-DIHYDRO-1,4-BENZODIOXIN-6-YL METHANESULFONATE;
3-[(METHYLAMINO)METHYL]-2,3-DIHYDRO-1,4-BENZODIOXIN-6-YL TRIFLUOROMETHANESULFONATE;
3-[(ETHYLAMINO)METHYL]-2,3-DIHYDRO-1,4-BENZODIOXIN-6-YL TRIFLUOROMETHANESULFONATE;
3-[(BUTYLAMINO)METHYL]-2,3-DIHYDRO-1,4-BENZODIOXIN-6-YL TRIFLUOROMETHANESULFONATE;
3-{[(2-HYDROXYETHYL)AMINO]METHYL}-2,3-DIHYDRO-1,4-BENZODIOXIN-6-YL TRIFLUOROMETHANESULFONATE;
3-[(DIPROPYLAMINO)METHYL]-2,3-DIHYDRO-1,4-BENZODIOXIN-6-YL TRIFLUOROMETHANESULFONATE;
3-{[ETHYL(PROPYL)AMINO]METHYL}-2,3-DIHYDRO-1,4-BENZODIOXIN-6-YL TRIFLUOROMETHANESULFONATE;
3-(PIPERIDIN-1-YLMETHYL)-2,3-DIHYDRO-1,4-BENZODIOXIN-6-YL TRIFLUOROMETHANESULFONATE;
3-[(DIMETHYLAMINO)METHYL]-2,3-DIHYDRO-1,4-BENZODIOXIN-6-YL TRIFLUOROMETHANESULFONATE;
3-(MORPHOLIN-4-YLMETHYL)-2,3-DIHYDRO-1,4-BENZODIOXIN-6-YL TRIFLUOROMETHANESULFONATE;
3-[(DIETHYLAMINO)METHYL]-2,3-DIHYDRO-1,4-BENZODIOXIN-6-YL TRIFLUOROMETHANESULFONATE;
3-(PYRROLIDIN-1-YLMETHYL)-2,3-DIHYDRO-1,4-BENZODIOXIN-6-YL TRIFLUOROMETHANESULFONATE;
3-[(ALLYLAMINO)METHYL]-2,3-DIHYDRO-1,4-BENZODIOXIN-6-YL TRIFLUOROMETHANESULFONATE;
3-{[ETHYL(METHYL)AMINO]METHYL}-2,3-DIHYDRO-1,4-BENZODIOXIN-6-YL TRIFLUOROMETHANESULFONATE;
3-{[(2-METHOXYETHYL)AMINO]METHYL}-2,3-DIHYDRO-1,4-BENZODIOXIN-6-YL TRIFLUOROMETHANESULFONATE;
3-(AZETIDIN-1-YLMETHYL)-2,3-DIHYDRO-1,4-BENZODIOXIN-6-YL TRIFLUOROMETHANESULFONATE;
3-{[METHYL(PROPYL)AMINO]METHYL}-2,3-DIHYDRO-1,4-BENZODIOXIN-6-YL TRIFLUOROMETHANESULFONATE;
1-[(6,7-DIFLUORO-2,3-DIHYDRO-1,4-BENZODIOXIN-2-YL)METHYL]PYRROLIDINE;
N-{[(2S)-7-(TRIFLUOROMETHYL)-2,3-DIHYDRO-1,4-BENZODIOXIN-2-YL]METHYL}PROPAN-1-AMINE;
1-(5,7-DIFLUORO-2,3-DIHYDRO-1,4-BENZODIOXIN-2-YL)-N-METHYLMETHANAMINE;
N-[(5,7-DIFLUORO-2,3-DIHYDRO-1,4-BENZODIOXIN-2-YL)METHYL]METHANAMINE;
N-[(5,7-DIFLUORO-2,3-DIHYDRO-1,4-BENZODIOXIN-2-YL)METHYL]PROP-2-EN-1-AMINE;
4-[(5,7-DIFLUORO-2,3-DIHYDRO-1,4-BENZODIOXIN-2-YL)METHYL]MORPHOLINE;
N-[(5,7-DIFLUORO-2,3-DIHYDRO-1,4-BENZODIOXIN-2-YL)METHYL]BUTAN-1-AMINE;
N-[(5,7-DIFLUORO-2,3-DIHYDRO-1,4-BENZODIOXIN-2-YL)METHYL]-N-PROPYLPROPAN-1-AMINE;
1-(5,7-DIFLUORO-2,3-DIHYDRO-1,4-BENZODIOXIN-2-YL)-N,N-DIMETHYLMETHANAMINE;
N-[(5,7-DIFLUORO-2,3-DIHYDRO-1,4-BENZODIOXIN-2-YL)METHYL]-N-ETHYLETHANAMINE;
N-[(5,7-DIFLUORO-2,3-DIHYDRO-1,4-BENZODIOXIN-2-YL)METHYL]PROPAN-2-AMINE;
N-[(5,7-DIFLUORO-2,3-DIHYDRO-1,4-BENZODIOXIN-2-YL)METHYL]-N-METHYLPROPAN-1-AMINE;
N-[(5,7-DIFLUORO-2,3-DIHYDRO-1,4-BENZODIOXIN-2-YL)METHYL]-N-ETHYLPROPAN-1-AMINE;
2-{[(5,7-DIFLUORO-2,3-DIHYDRO-1,4-BENZODIOXIN-2-YL)METHYL]AMINO}ETHANOL;
N-[(5,7-DIFLUORO-2,3-DIHYDRO-1,4-BENZODIOXIN-2-YL)METHYL]-N-METHYLETHANAMINE;
N-[(5,7-DIFLUORO-2,3-DIHYDRO-1,4-BENZODIOXIN-2-YL)METHYL]-2-METHOXYETHANAMINE;
1-[(5,7-DIFLUORO-2,3-DIHYDRO-1,4-BENZODIOXIN-2-YL)METHYL]AZETIDINE;
N-[(5,7-DIFLUORO-2,3-DIHYDRO-1,4-BENZODIOXIN-2-YL)METHYL]-2-METHYLPROPAN-1-AMINE;
1-[(5,7-DIFLUORO-2,3-DIHYDRO-1,4-BENZODIOXIN-2-YL)METHYL]PYRROLIDINE;
1-[(5,7-DIFLUORO-2,3-DIHYDRO-1,4-BENZODIOXIN-2-YL)METHYL]PIPERIDINE;
N-[(5,7-DIFLUORO-2,3-DIHYDRO-1,4-BENZODIOXIN-2-YL)METHYL]-3-FLUOROPROPAN-1-AMINE;
2-({[(2S)-7-(TRIFLUOROMETHYL)-2,3-DIHYDRO-1,4-BENZODIOXIN-2-YL]METHYL}AMINO)ETHANOL; or
N-{[7-(FLUOROMETHYLSULFONYL)-3,4-DIHYDRO-2H-CHROMEN-2-YL]METHYL}-N-PROPAN-1-AMINE;
any of its stereoisomers or any mixture of its stereoisomers, or an N-oxide thereof; or a pharmaceutically acceptable salt thereof.

11. A pharmaceutical composition, comprising a therapeutically effective amount of a compound of claim 1, any of its stereoisomers or any mixture of its stereoisomers, or an N-oxide thereof, or a pharmaceutically acceptable salt thereof, together with at least one pharmaceutically acceptable carrier, excipient or diluent.

12. A compound according to claim 1, any of its stereoisomers or any mixture of its stereoisomers, or an N-oxide thereof, or a pharmaceutically acceptable salt thereof, for use as a medicament.

13. A compound according to claim 1, any of its stereoisomers or any mixture of its stereoisomers, or an N-oxide thereof, or a pharmaceutically acceptable salt thereof, for use in the treatment, prevention or alleviation of a disease or a disorder or a condition of a mammal, including a human, which disease, disorder or condition is responsive to modulation of dopaminergic function in the central nervous system.

14. (canceled)

15. (canceled)

16. (canceled)

17. A method for treatment, prevention or alleviation of a disease or a disorder or a condition of a living animal body, including a human, which disorder, disease or condition is responsive to modulation of dopaminergic function in the central nervous system, which method comprises the step of administering to such a living animal body in need thereof a therapeutically effective amount of a compound according to claim 1, or any of its stereoisomers or any mixture of its stereoisomers, or an N-oxide thereof; or a pharmaceutically acceptable salt thereof.

18. The method according to claim 17, wherein the disease, disorder or condition is movement disorders, Parkinson's disease, Parkinsonism, dyskinesias, L-DOPA induced dyskinesias, dystonias, tics, tremor, Huntington's disease, iatrogenic psychoses and hallucinoses, non-iatrogenic psychoses and hallucinoses, schizophrenia, schizophreniform disorders, bipolar disorder, mood disorders, anxiety disorders, depression, obsessive-compulsive disease, neurodevelopmental disorders, Autism spectrum disorders, ADHD, Cerebral Palsy, Gilles de la Tourette's syndrome, neurodegenerative disorders, dementia, age-related cognitive impairment, sleep disorders, sexual disorders, eating disorders, obesitas, headaches, pains in conditions characterized by increased muscular tone, substance abuse, Alzheimer's disease or dementia disorders related to Alzheimer's disease.
